(19) United States
(12) Patent Application Publication
(10) Pub. No.: US 2018/0153637 A1
(43) Pub. Date: Jun. 7, 2018

(54) SURGICAL GAS SHIELD MODULE WITH ELEVATION SYSTEM

(71) Applicants: Modher Ibrahim Al-Shawi, Bulleen, Victoria (AU); Ibrahim Mudhir Ahmed Al-Shawi, Amman (JO)

(72) Inventors: Modher Ibrahim Al-Shawi, Bulleen, Victoria (AU); Ibrahim Mudhir Ahmed Al-Shawi, Amman (JO)

(21) Appl. No.: 15/575,343

(22) PCT Filed: May 26, 2016
(86) PCT No.: PCT/IB2016/053097

§ 371 Date: Nov. 18, 2017

Related U.S. Application Data
(60) Provisional application No. 62/171,357, filed on Jun. 5, 2015.
(60) Provisional application No. 62/203,550, filed on Aug. 11, 2015.
(60) Provisional application No. 62/305,558, filed on Mar. 9, 2016.

(51) Int. Cl. A61B 90/40; A61M 1/00

(52) U.S. Cl. CPC A61B 90/40; A61M 1/0088; A61B 2218/008; A61B 2217/005; A61B 2090/401

(57) ABSTRACT

A surgical gas shield device comprising a gas shield module and an elevation system. The gas shield module has a body housing which is divided into a discharge chamber and a suction chamber. The elevation system is configured to hold said gas shield module around a surgical site at a desired position and orientation. Discharge of a gas introduced from a positive pressure source through discharge ports of the gas shield module over the surgical site and suction of the gas and other air borne byproducts from the surgical site through the suction ports of the gas shield device by application of a partial vacuum create a gas shield over the surgical site. The gas shield module can be fork type or closed loop type. The elevation system can be flexible fold type or inflatable type.

### CROSS REFERENCE TO RELATED APPLICATIONS

This Non-Provisional Utility Patent Application claims the benefit of the filing dates of U.S. Provisional Patent Application Ser. No. 62/171,357, filed on Jun. 5, 2015, U.S. Provisional Patent Application Ser. No. 62/203,550, filed on Aug. 11, 2015, and U.S. Provisional Patent Application Ser. No. 62/305,558, filed on Mar. 9, 2016, all of which are herein incorporated by reference.

### TECHNICAL FIELD

The present invention generally relates to systems, devices and methods for assisting in evacuation of surgical by-products, such as plume, fumes, smoke, contaminants, odorous gases, living cells and viruses produced at a surgical site during electrosurgery (diathermy), laser beam, and ultrasonic device assisted surgery. More specifically, the present invention relates to system and method for an easy to use, efficient surgical gas shield which can be placed at a desired position based on the dimensional and topographical features of the surgical site.

### BACKGROUND ART

At surgery sites, for protecting surgeons and operating room staff from diathermy or laser fumes, as well as other contaminants emanating from a surgical site, gas shields are used. The gas shields presently available are neither easy to use nor efficient in removing the contaminants and fumes from a surgical site. The existing gas shield devices sometime do not allow the surgeons to work easily on the surgery site. This is particularly so when a wide incision is required as the existing surgical shield devices make a closed loop around the surgery site. Another problem associated with most of the existing gas shield devices is that these devices evacuate the contaminants and fumes from a surgery site by use of vacuum and, in doing so, air from the body cavity at the surgery site gets drawn out and, also, localized evaporative cooling may occur at the surgery site. Another problem with existing devices is that some of them require a dedicated assistant to place the device in the desired location. In case of some surgeries, there may be certain factors such as topography of the surgery site and/or need to operate surgical devices etc. which necessitate the gas shield to be positioned little above the surgical site. For the same reasons the surgical gas shield may be required to be positioned at different plane of inclination. The existing gas shield devices do not address these problems.

Thus, there is a need to provide a surgical gas shield which is efficient, easy to use, gives surgeons' easy access to surgery site and can be positioned at a surgical site as per requirement.

### OBJECTS OF THE INVENTION

An object of the present invention is to provide a portable surgical gas shield device which is efficient yet cost effective.

Another object of the present invention is to provide a surgical gas shield device which offers easy access to and better view of a surgery site.

Yet another object of the present invention is to provide an elevation system for a surgical gas shield device which can support non-uniform weight distribution of surgical gas shield module mounted on the elevation system.

Still another object of the present invention is to provide a surgical gas shield device which can be positioned at a plane elevated from the site of surgery parallel or inclined to the plane of surgery site at any desired angle.

Another object of the present invention is to provide a surgical gas shield device having a convenient mechanism for regulating gas pressure to adjust the elevation of the gas shield device from the surgery site.

Still another object of the present invention is to provide a surgical gas shield device having a single valve for gas flow control to inflating and deflating the elevation system of the device as per requirement.

Yet another object of the present invention is to provide a surgical gas shield device comprising a flexible expandable collapsible structure as support for elevating and maintaining the gas shield at a desired position.

Another object of the present invention is to provide a surgical gas shield device which can be positioned over any uneven body contour.

A further object of the present invention is to provide a surgical gas shield device having a non-inflatable type elevation system which is inexpensive yet convenient to use and effective.

Another object of the present invention is to provide a surgical gas shield device which stays stable on site free from push or pull which may be exerted by the suction/discharge tube during raising or lowering of the system or by gas pressure change.

Details of the foregoing objects and of the invention, as well as additional objects, features and advantages of the invention will become apparent to those skilled in the art upon consideration of the following detailed description of the preferred embodiments exemplifying the best mode of carrying out the invention as presently perceived.

### BRIEF SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

The present invention is directed to a system and method for providing a shield or curtain of gas at a surgery site (also referred to as “surgical site” alternatively and interchangeably) to protect surgeons, operating staff and the patient from fumes and other contaminants generated during operating procedure such as diathermy, electrosurgery or laser. In a preferred embodiment, the discharge side of the gas shield module is configured to be connected to a positive pressure gas source while the suction side of the gas shield module can be connected to a negative pressure source. In some embodiments the gas shield module is configured to be operable in suction only mode. The surgical gas shield device of the present invention comprises a gas shield module to produce the gas shield and an elevation system which enables positioning of the gas shield module at a desired elevation and inclination. The elevation system also enables placement of the gas shield module over any uneven body part of a patient. The present invention proposes several different embodiments for the gas shield module and the elevation system and, accordingly, the surgical gas shield device also has a number of embodiments. The gas shield module can be of a tuning fork type shape or it can be of a closed loop shape. The elevation system can be inflatable type or it can be a zigzag shaped flexible fold type which is not inflatable. The gas shield module which is mounted on an inflatable elevation system requires one or more regulating valves whereas no such valve or regulator is required in the gas shield module mounted on a zigzag shaped flexible fold elevation system. In different embodiments of the flexible fold elevation system, one or more different types of positioner mechanisms are provided which enable adjustment of the height and elevation of a gas shield module mounted on the elevation system as per requirement. Optionally, an anchorage mechanism can be provided in the surgical gas shield device using inflatable elevation system to isolate the surgical gas shield device from the push or pull that may be exerted by the positive pressure and negative pressure tubes connected to the gas shield module.

To the accomplishment of the foregoing and related ends, certain illustrative aspects of the disclosed invention are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF DRAWINGS

In order to describe the manner in which features and other aspects of the present disclosure can be obtained, a more particular description of certain subject matter will be rendered by reference to specific embodiments which are illustrated in the appended drawings. Understanding that these drawings depict only typical embodiments and are not therefore to be considered to be limiting in scope, nor drawn to scale for all embodiments, various embodiments will be described and explained with additional specificity and details through the use of the accompanying drawings in which:

FIG. 1A illustrates a surgical gas shield device in accordance with a first embodiment of the present invention;

FIG. 1B illustrates a surgical gas shield device at elevated condition in accordance with the first embodiment of the present invention;

FIG. 2A illustrates an exploded view of a surgical gas shield device in accordance with the first embodiment of the present invention;

FIG. 2B illustrates an exploded view of a surgical gas shield device showing the elevation system in inflated condition in accordance with the first embodiment of the present invention;

FIG. 3A illustrates a surgical gas shield device in accordance with a second embodiment of the present invention;

FIG. 3B illustrates a surgical gas shield device with the elevation system in expanded condition in accordance with the second embodiment of the present invention;

FIG. 3C illustrates a surgical gas shield device with the elevation system having an elevation adjustment mechanism (also referred to as positioner mechanism) in accordance with the second embodiment of the present invention;

FIG. 4A illustrates a perspective view of the gas shield module of the surgical gas shield device in accordance with the first embodiment of the present invention;

FIG. 4B illustrates top half or top wall of a gas shield module of the surgical gas shield device in accordance with the first embodiment of the present invention;

FIG. 4C illustrates bottom half or a bottom wall of a gas shield module of the surgical gas shield device in accordance with the first embodiment of the present invention;

FIG. 5A illustrates a top sectional view of gas shield module of the surgical gas shield device in accordance with the second embodiment of the present invention showing the flow channels and flow directions;

FIG. 5B illustrates a section of the discharge chamber of a gas shield module of the surgical gas shield device in accordance with the first and second embodiments of the present invention;

FIG. 5C illustrates a section of the suction chamber of a gas shield module of the surgical gas shield device in accordance with the first and second embodiments of the present invention;

FIG. 6A illustrates a loop type gas shield module of the surgical gas shield device in accordance with an embodiment of the present invention;

FIG. 6B illustrates a top sectional view of loop type gas shield module of the surgical gas shield device in accordance with an embodiment of the present invention;

FIG. 6C illustrates a top sectional view of another embodiment of a loop type gas shield module of the surgical gas shield device;

FIG. 6D illustrates a top sectional view of a loop type gas shield module of the surgical gas shield device showing gas flow directions in accordance with an embodiment of the present invention;

FIG. 7 illustrates a top sectional view of a suction only gas shield module of the surgical gas shield device in accordance with the second embodiment of the present invention;

FIG. 8A illustrates a suction only loop type gas shield module of the surgical gas shield device in accordance with an embodiment of the present invention;

FIG. 8B illustrates a top sectional view of suction only loop type gas shield module of the surgical gas shield device in accordance with an embodiment of the present invention;

FIG. 8C illustrates a top sectional view of suction only another embodiment of a loop type gas shield module of the surgical gas shield device;

FIG. 8D illustrates a top sectional view of a suction only loop type gas shield module of the surgical gas shield device showing gas flow directions in accordance with an embodiment of the present invention;

FIG. 9A through FIG. 9H illustrate various embodiments of the gas shield module of the surgical gas shield device of the present invention;

FIG. 10A illustrates a bottom perspective view of a fork type gas shield module of the surgical gas shield device in accordance with the first embodiment of the present invention;

FIG. 10B illustrates a top perspective view of an inflatable elevation system of the surgical gas shield device in accordance with an embodiment of the present invention;

FIG. 10C illustrates a top perspective view of stacking arrangement of another embodiment of the inflatable elevation system of the surgical gas shield device;

FIG. 10D illustrates a cross-sectional view of the inflatable elevation system gas cushion element in deflated state in accordance with an embodiment of the present invention;

FIG. 10E illustrates a cross-sectional view of the inflatable elevation system gas cushion element in partially inflated state in accordance with an embodiment of the present invention;

FIG. 11A is a perspective view of a push type valve used with the first embodiment of the present invention for regulating gas flow to inflate or deflate gas cushion elements of an inflatable elevation system;

FIG. 11B is an exploded view of the push type valve of FIG. 11A in accordance with an embodiment of the present invention;

FIG. 11C illustrates a section of the gas shield module of first embodiment with the push type valve of FIG. 11A at idle condition in accordance with an embodiment of the present invention;

FIG. 11D illustrates a section of the gas shield module of the first embodiment with the push type valve of FIG. 11A at fully actuated condition for inflating an inflatable type elevation system;

FIG. 11E illustrates a section of the gas shield module of the first embodiment with the push type valve of FIG. 11A at partially actuated condition for deflating an inflatable type elevation system;

FIG. 12A illustrates a perspective view of a rocker type valve comprising a torsion spring for use in the gas shield module of first embodiment;

FIG. 12B illustrates a perspective view of a rocker type valve of FIG. 12A comprising a compression spring for use in the gas shield module of first embodiment;

FIG. 12C is a bottom perspective view of a gas shield module of first embodiment fitted with a rocker type valve of FIG. 12A;

FIG. 13A is a sectional view of a part of a gas shield module with the rocker type valve in idle state in accordance with an embodiment of the present invention;

FIG. 13B is a sectional view of a part of a gas shield module of first embodiment with the rocker type valve in gas cushion inflate mode in accordance with an embodiment of the present invention;

FIG. 13C is a sectional view of a part of a gas shield module with the rocker type valve in gas cushion deflate mode in accordance with an embodiment of the present invention;

FIG. 14A illustrates a perspective view of a surgical gas shield device having twin regulating valves for controlling the elevation produced by the inflatable elevation system in accordance with an embodiment of the present invention;

FIG. 14B illustrates a side view of a surgical gas shield device having twin regulating valves in accordance with an embodiment of the present invention;

FIG. 15 illustrates an exploded perspective view of a surgical gas shield device having an elevation system with twin regulating valves in accordance with an embodiment of the present invention;

FIG. 16A illustrates an exploded view of one of the twin regulating valves for the gas shield module of first embodiment;

FIG. 16B illustrates front view of the valve of FIG. 16A;

FIG. 16C illustrates front view of the valve of FIG. 16A showing position of a compression spring in accordance with an embodiment of the present invention;

FIG. 16D illustrates a perspective view of a section of the gas shield module of first embodiment having twin regulating valves in accordance with an embodiment of the present invention;

FIG. 16E illustrates a perspective view of a section of the gas shield module of the surgical gas shield device showing one of the twin regulating valves in action in accordance with an embodiment of the present invention;

FIG. 17A illustrates another embodiment of one of the twin regulating valves of a gas shield module in accordance with an embodiment of the present invention;

FIG. 17B illustrates exploded view of the valve of FIG. 17A in accordance with an embodiment of the present invention;

FIG. 17C illustrates use of adhesive sheet with a surgical gas shield device in accordance with an embodiment of the present invention;

FIG. 18A illustrates an inflated elevation system holding the gas shield module at a raised position in accordance with an embodiment of the surgical gas shield device of the present invention;

FIG. 18B illustrates perspective view of an anchorage mechanism for isolating the surgical gas shield device module from the push or pull forces which may be exerted by the pressure tube and the evacuation tube on the device;

FIG. 18C illustrates exploded view of the anchorage mechanism for isolating the assembly of gas shield module and elevation system from the push or pull forces which may be exerted by the pressure tube and the evacuation tube on the assembly in accordance with an embodiment of the present invention;

FIG. 19A illustrates flexible folds of flexible fold type elevation system in accordance with an embodiment of the present invention;

FIG. 19B illustrates a section of the first embodiment of the flexible fold elevation system comprising a positioner mechanism having one or more positioner strips for controlling the elevation and inclination of the flexible folds in accordance with an embodiment of the present invention;

FIG. 20A illustrates a positioner strip used in elevation system of FIG. 19B for controlling the elevation and inclination of the flexible folds in accordance with an embodiment of the present invention;

FIG. 20B illustrates a positioner mechanism used in elevation system of FIG. 19B for controlling the elevation and inclination of the flexible folds in accordance with an embodiment of the present invention;

FIG. 20C illustrates an exploded view of the positioner mechanism of FIG. 20B in accordance with an embodiment of the present invention;

FIG. 20D illustrates a bottom perspective view of the surgical gas shield device comprising a first embodiment of the flexible fold type elevation system;

FIG. 20E illustrates a top perspective view of the surgical gas shield device comprising the first embodiment flexible fold type elevation system;

FIG. 21A illustrates a second embodiment of the flexible fold type elevation system having a positioner mechanism for controlling the elevation and inclination of the flexible folds in accordance with an embodiment of the present invention;

FIG. 21B illustrates a third embodiment of the flexible fold type elevation system having a mechanism for controlling the elevation and inclination of the flexible folds in accordance with an embodiment of the present invention;

FIG. 21C illustrates a fourth embodiment of the flexible fold type elevation system having a mechanism for controlling the elevation and inclination of the flexible folds in accordance with an embodiment of the present invention;

FIG. 21D illustrates a fifth embodiment of the flexible fold type elevation system having a mechanism for controlling the elevation and inclination of the flexible folds in accordance with an embodiment of the present invention;

FIG. 21E illustrates a sixth embodiment of the flexible fold type elevation system having a mechanism for controlling the elevation and inclination of the flexible folds in accordance with an embodiment of the present invention;

FIG. 22A illustrates the surgical gas shield device comprising an elevation system having one or more adhesive strips or tapes or fasteners or other means of attachment or re-attachment in accordance with an embodiment of the present invention;

FIG. 22B is a top view illustrating a possible shape of the flexible fold type elevation system to enable positioning of gas shield module at a desired rest position while accommodating the variations in weight distribution of the gas shield module in accordance with an embodiment of the present invention;

FIG. 23A illustrates a gas shield module of a surgical gas shield device positioned at an inclined plane with the help of a flexible fold elevation system in accordance with an embodiment of the present invention;

FIG. 23B illustrates a gas shield module of a surgical gas shield device positioned at another inclined plane with the help of a flexible fold elevation system in accordance with an embodiment of the present invention; and

FIG. 24 illustrates surgical gas shield devices of various dimensions positioned on uneven contours of a patient's body.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures and components have not been described in detail so as not to obscure the present invention.

Embodiments of the present invention are described herein in the context of a system and method for providing a gas shield at a surgery site through a surgical gas shield device. The surgical gas shield device of the present invention can be positioned at an elevated place to suit the topographical and surgical requirements at a surgery site. A primary aim of the present invention is to protect surgeon and/or staff from diathermy or laser fumes. Other potential benefits include protecting operating room staff from contamination from the patient and to protect the patient from operating room borne contaminants.

The surgical gas shield device of the present invention comprises a gas shield module and an elevation system. The following detailed description refers to several embodiments of the surgical gas shield device of the present invention. Those of ordinary skill in the art will realize that the following detailed description of the present invention is illustrative only and is not intended to be in any way limiting. Other embodiments of the present invention will readily suggest themselves to such skilled persons having the benefit of this disclosure. For example, numerous embodiments of the surgical gas shield device of the present invention comprising different combinations of various embodiments of the gas shield module and elevation system may be consistent with the spirit of the invention herein. Reference will now be made in detail to implementations of the present invention as illustrated in the accompanying drawings. The same reference indicators will be used throughout the drawings and the following detailed description to refer to the same or like parts.

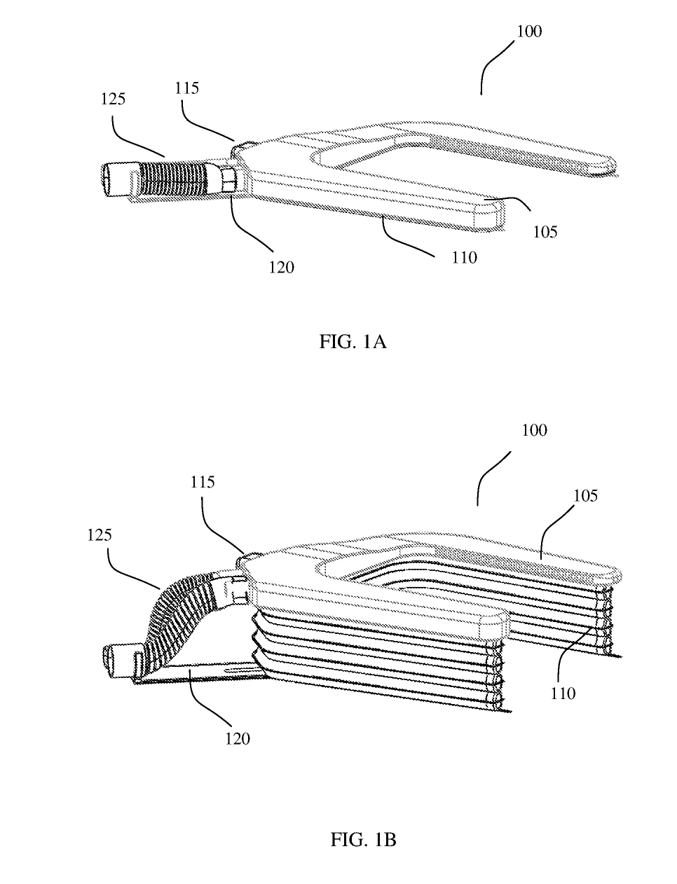

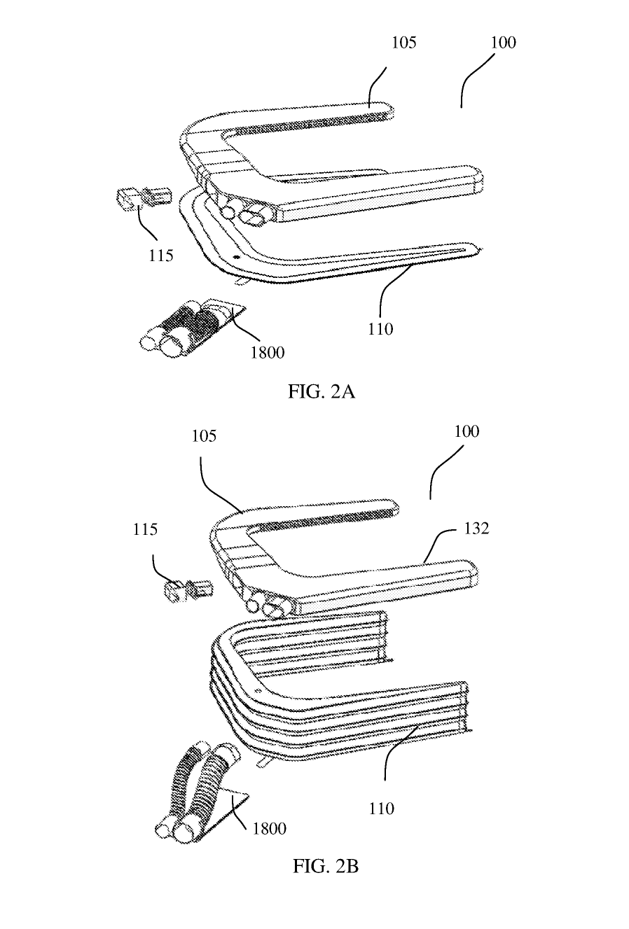

FIGS. 1A, 1B, 2A & 2B show first embodiment 100 of the surgical gas shield device of the present invention. The first embodiment device 100 comprises a gas shield module 105 and an elevation system 110. The gas shield module 105 removes air borne byproducts such as e.g. fumes, toxic gases and vapors including benzene, hydrogen cyanide, formaldehyde, viruses and other contaminants produced during surgical procedures which use Lasers, diathermy, ultrasonic generators etc. The elevation system 110, on which the gas shield module 105 is mounted, facilitates positioning of the gas shield module 105 at a plane elevated parallel or inclined to the plane of surgery site at any desired height. The surgical gas shield module 105 further comprises one or more regulating valves 115 and an anchorage system 1800. The anchorage system 1800 includes one or more flexible tubes 125.

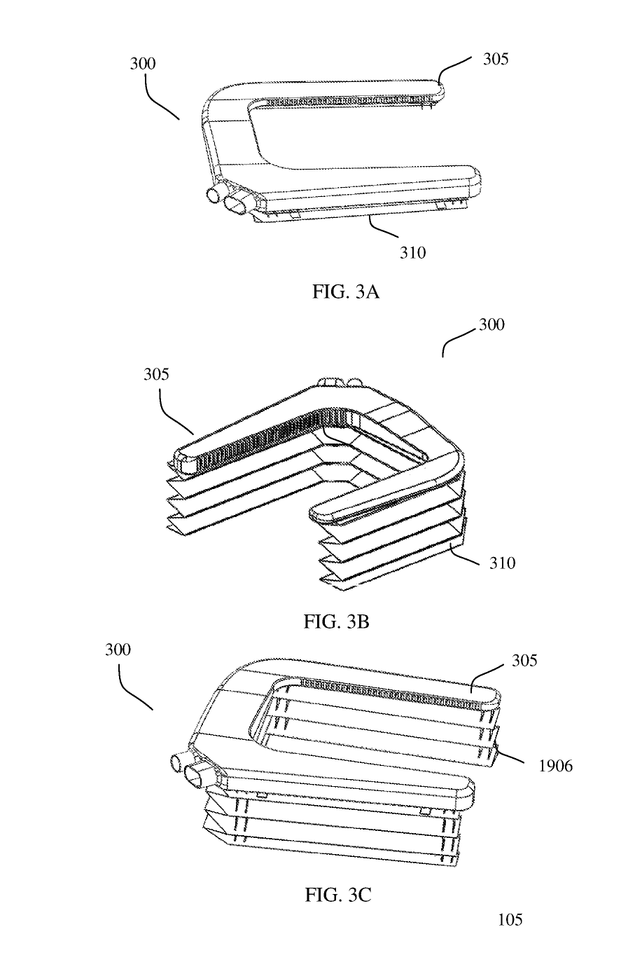

FIGS. 3A, 3B & 3C show a second embodiment 300 of the surgical gas shield device of the present invention. The second embodiment device 300 differs from the first embodiment device 100 mainly in terms of the elevation system used. In the second embodiment device 300, the elevation system 310 comprises two or more zigzag-shaped or z-shaped paddings or flexible folds or bellows which are not inflatable unlike the elevation system 110. Consequently, the gas shield module 305 does not require regulating valves which are required in the elevation system 110 of the first embodiment device 100.

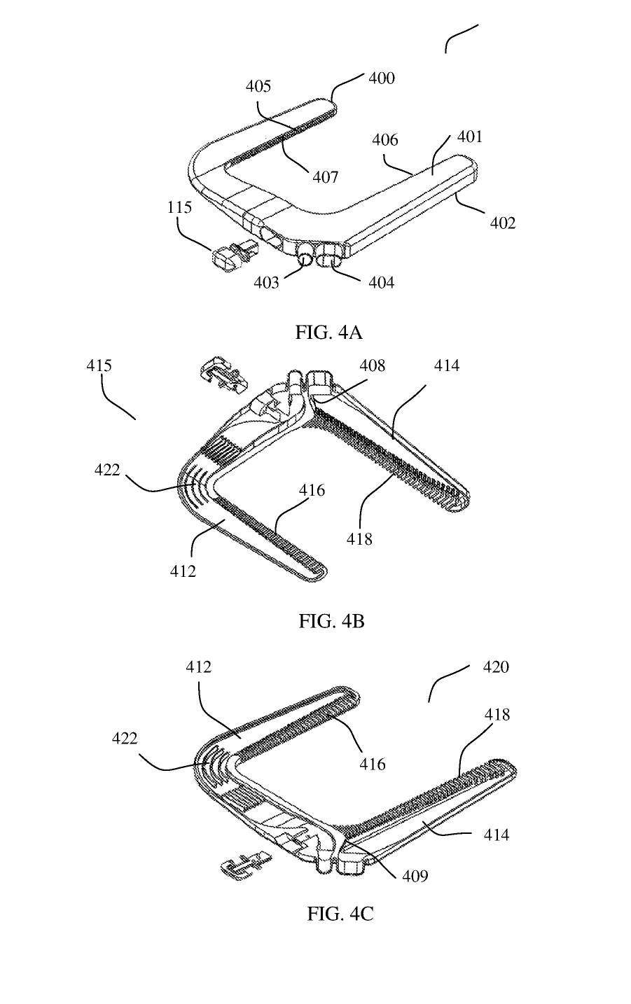

FIG. 4A shows a gas shield module 105 from the first embodiment device 100. According to an embodiment of the present invention, the gas shield module may be formed in an open fork shape as well as in a closed loop shape. In a preferred embodiment, a fork-shaped surgical air shield allows the air shield to be more easily adapted to the topography of a patient's body as well as providing better access to the surgical site by the surgeon. This adaptability is especially important when the surgical gas shield device is secured to an incision site at an area of the body that is not flat, such as would be commonly encountered during surgery on a breast, knee, neck, leg, or arm. Except the presence of one or more regulating valves 115 in gas shield module 105, the fork type gas shield modules 105 and 305 are similar in structure and are alternatively and collectively referred to as tuning-fork type gas shield module or fork type gas shield module. The fork type gas shield module 105 has a body housing 400 which includes a top wall 401 and a bottom wall 402. In a preferred embodiment, the body housing 400 is made from rigid or semi rigid surgical grade plastic such as ABS, Nylon, PVC or any other suitable material which can provide structural rigidity without collapsing under vacuum. The top wall 401 and the bottom wall 402 are configured, when joined together, to define two chambers 412 and 414 separated from each other by complementary protruding sections 408 and 409 (shown in FIG. 4B and FIG. 4C) disposed inside the walls 401 and 402 respectively. The chamber 412 (hereinafter referred to as discharge chamber 412) is provided with an inlet 403 adaptable for coupling with an external pressurized gas supply tube connected to a positive pressure source. Similarly, the chamber 414 (hereinafter referred to as suction chamber 414) is provided with an outlet 404 adaptable for coupling with an external suction tube connected to a negative pressure source capable of creating partial vacuum. Optionally, a flow regulating valve 115 is disposed on the chamber 412.

FIG. 4B and FIG. 4C show the fork type gas shield module 105 in two halves—top half 415 and bottom half 420. As shown in FIG. 4B and FIG. 4C, the discharge chamber 412 and the suction chamber 414 are provided with multiple straight and/or arcuate/shaped/arched projections 422 on the inner sides so that, when assembled with the inner sides facing each other, the projections 422 on the opposite halves abut and complement each other to form one or more flow channels.

A plurality of projections 416 disposed on both the walls 401 and 402 on the inner periphery 405 of the discharge chamber 412 define a plurality of discharge ports 407 (407 shown in FIG. 4A). Similarly, on the opposite side of 405, on inner periphery 406, a plurality of projections 418 are disposed on both the walls 401 and 402 of the suction chamber 414 which define a plurality of suction ports.

As shown in FIGS. 4A, 4B and 4C, the housing body 400 is configured to accommodate the flow regulating valve 115 in the discharge chamber 412 downstream to the inlet 403 in the gas flow direction. Mirror image projections on both the walls 401 and 402 abut to form one or more flow channels which facilitate regulation of gas flow through the discharge chamber 412 for inflating or deflating an elevation system 110 (110 shown in FIG. 2B) with the help of the regulating valve 115.

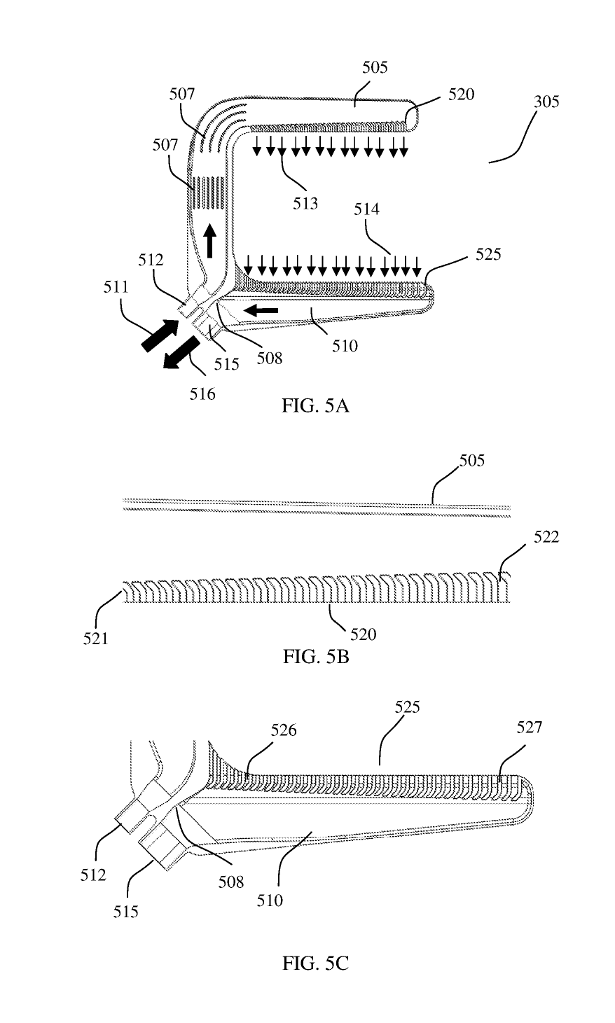

FIG. 5A illustrates a top sectional view of the gas shield module 305 of the second embodiment device 300. As mentioned above, structurally both the gas shield modules 105 and 305 are the same except that the gas shield module 305 does not need the gas regulating valve 115 of the gas shield module 105 and, accordingly, the discharge chamber 505 does not have the special flow channel arrangement which the discharge chamber 412 has for the regulating valve 115 in the gas shield module 105. Also, no vent or aperture is required in the gas shield module 305 for transmission of fluid to and from the elevation system which is required in case of the gas shield module 105. The pressurized gas (air, nitrogen, carbon dioxide or any suitable sterile gas) enters the discharge chamber 505 from an external supply (not shown in the drawings) detachably attached to the inlet 512 in the flow direction 511. The flow channels 507 disposed inside the discharge chamber 505 facilitate and guide the flow of gas with minimum pressure drop from inlet 512 to the discharge ports 520. The pressurized gas then exits through the plurality of discharge ports 520 in the direction 513 and flows over the surgical site around which the surgical gas shield device of the present invention is placed. FIG. 5B illustrates a section of the discharge chamber 505 with enlarged view of the plurality of discharge ports 520. In a preferred embodiment, each of the individual ports of the discharge ports 520 comprises a curved wall section at the gas entry point and a straight wall section at the gas exit point so that the gas gets discharged through the discharge ports 520 in a direction 513 which is substantially perpendicular to the row of the discharge ports 520. In another embodiment, the size of discharge ports 520 increases in a gradual manner from the proximal end 521 to the distal end 522 of the discharge chamber 505 to maintain a uniform or a desired pattern of flow characteristics of the flowing gas at the discharge ports by compensating pressure drop.

The suction chamber 510, which is completely separated/isolated from the discharge chamber 505 by a wall 508, is detachably connected through the outlet 515 with an external evacuation chamber (not shown in the drawings) maintained at a negative pressure or partial vacuum. Consequently, the suction chamber 510 remains at a negative pressure and tend to suck in the gas and other air borne byproducts of surgical procedures from outside through the plurality of suction ports 525 in the direction 514. In a preferred embodiment, each of the individual ports of the suction ports 525 comprises a straight wall section at the gas entry point from outside and a curved wall section at the gas exit point inside the suction chamber 510 toward the outlet 515 so that the gas gets sucked in through the suction ports 520 in a direction 514. In another embodiment, as shown in FIG. 5C, the size of suction ports 525 increases in a gradual manner from the proximal end 526 to the distal end 527 of the suction chamber 505 to maintain a uniform or a desired pattern of flow characteristics of the flowing gas at the suction ports by compensating pressure drop. Also, preferably, the overall size or volume of the suction chamber 510 is made bigger than the size or volume of the section of the discharge chamber 505 having the discharge ports 520. The overall suction area offered by the suction ports 525 can also be made more than the overall discharge area offered by the discharge ports 520. The suction ports 525 suck in atmospheric gases and air borne byproducts of surgery from the surgery site in addition to the gas discharged through the discharge ports 520. A bigger suction chamber 525 having greater suction area facilitates evacuation of this increased volume of gas and particles. The flow of gas from the discharge ports 520 to the suction ports 525 results in creation of a gas shield or gas curtain or gas barrier between the surgery site and the space above the gas shield on a plane roughly parallel to the plane of surgery site. The suction ports 525 remove the gas exiting through the discharge ports 520 together with any air borne byproducts of surgery such as smoke and particulates entrained by the stream of gas from the surgery site.

In the above description only one pattern for the air/gas flow channels is discussed with respect to the drawings. However, it will be obvious to any person skilled in the art that other patterns and shapes of flow channels are also possible. The primary aim of the design is to achieve a desired flow pattern, such as equal air velocities at the discharge/suction ports or a velocity profile across the surgical site as desired. Tapers and/or shields can be added to the multitude of gas jet supply and suction ports to accommodate performance requirements of the air curtain produced and to prevent ingress of fluids into the surgical cavity. Factors affecting performance and dimensions of the air shield module include, but are not limited to, the absolute and the relative velocities at the supply/discharge ports and exhaust/suction ports, whether the air flow jets are laminar or turbulent, the angle of any tilt in those ports due to bending following the outer contours of the patients body, module and slot thickness as well as the supply and exhaust pressures and maximum flow rates that can be accommodated by the pressure and suction pump(s) used. Another design factor is the “Force Number” which is defined as the square root of the ratio of the momentum force of the fluid layer at its source of origin to the pressure force across the layer as it passes over the opening or area plane to be protected. An important factor to be considered in the detailed design of the gas shield module of the present invention is the effect of the interference caused by operating surgeons' hands, incisions retractors and tools used in the procedure. The proportions of the cavity can be varied to accommodate the surgical cavity and are partially dictated by air curtain performance governing parameters. The surgical gas shield device is intended to be produced in several sizes to suit the incision size. Incision sizes typically vary between 5 cm and 25 cm. Each size has suitably sized discharge and suction ports to accommodate the resulting air flow rates without excessive pressure drop. In one embodiment, the surgical gas shield device is designed to accommodate what is known as the Alexis Port that surrounds the surgical wound so that the air shield module is an integral or a retrofitting component of the Alexis Port.

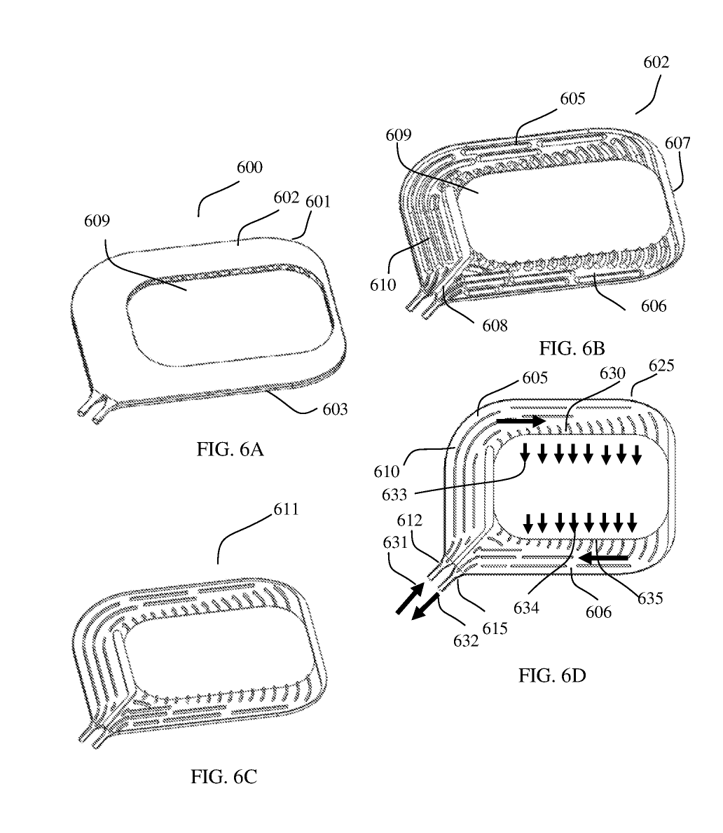

FIG. 6A illustrates a loop type (or closed loop type) gas shield module 600 which operates on the same working principle on which fork type gas shield modules 105 or 305 work depending on the selection of elevation system used with gas shield module. The loop type gas shield module 600 has a body housing 601 which includes a top wall 602 and a bottom wall 603. The top wall 602 and the bottom wall 603 are configured to define, when joined together, a discharge chamber 605 and a suction chamber 606 separated from each other by a protruding section 608 disposed inside the wall 602 as shown in FIG. 6B. FIG. 6C illustrates another embodiment 611 of the gas shield module 600 which has a different inside flow channel arrangement. Structurally the loop type gas shield module 600 is similar to the fork type gas shield modules 105 or 305 with the difference that, unlike the fork type gas shield modules, in the loop type gas shield module 600 the discharge chamber 605 and the suction chamber 606 meet each other physically (but not in fluid communication) at the distal end of the gas shield module 600 to form a cavity 609. As shown in FIG. 6D, the pressurized gas (air, nitrogen, carbon dioxide or any suitable sterile gas) enters the discharge chamber 605 from an external supply (not shown in the drawings) detachably attached to the inlet 612 in the flow direction 631. The flow channels 610 disposed inside the discharge chamber 605 facilitate and guide the flow of gas with minimum pressure drop from inlet 612 to the discharge ports 630. The pressurized gas then exits through the plurality of discharge ports 630 in the direction 633 and flows over the surgical site around which the surgical gas shield device of the present invention is placed. The suction chamber 606, which is completely separated/isolated from the discharge chamber 605 by a partition wall 608, is detachably connected through the outlet 615 with an external evacuation chamber (not shown in the drawings) maintained at a negative pressure or partial vacuum. Consequently, the suction chamber 606 remains at a negative pressure and tend to suck in the gas and other air borne byproducts of surgical procedures from outside through the plurality of suction ports 635 in the direction 634. In a preferred embodiment, each of the individual ports of the suction and discharge ports are designed to provide a smooth flow path for the discharged and sucked gases.

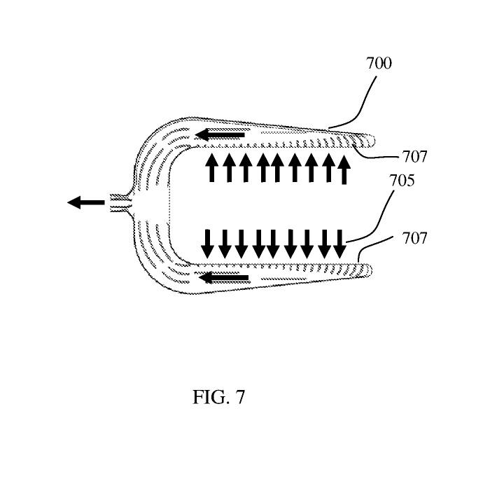

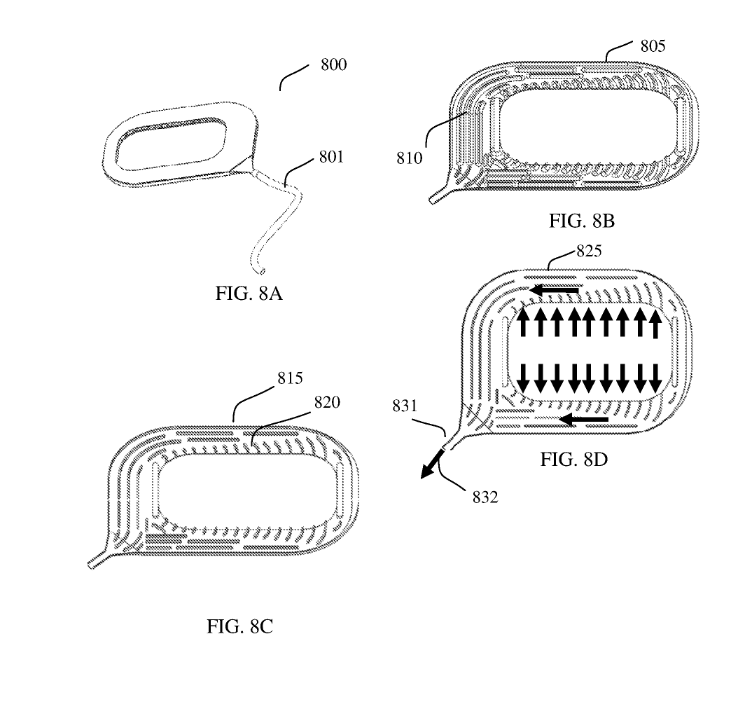

According to an embodiment of the present invention, reference to FIG. 7 and FIG. 8A through FIG. 8D, the gas shield module of the present invention may be configured to operate only with a suction or vacuum function (hereinafter referred to as suction only gas shield module). The suction only gas shield module has a single chamber which is used for drawing the air borne particles and gases into the gas shield module for evacuation through a single outlet. The suction only gas shield module offers the advantages of being usable with the air suction systems commonly found in modern operating rooms and eliminating the need to employ a separate device to produce the required pressurized gas. In this embodiment, only suction is produced at each of the ports (ports 707 of gas shield module 600 in FIG. 6 with suction in the direction 705, for example) of the surgical air shield. Additionally, this embodiment of the gas shield module is simplified, as it only requires one tube (tube 801 of gas shield module 800 in FIG. 8A for example) for suction, as opposed to the dual tube set-up required of the other primary embodiment. In the suction only gas shield module, a suction line (801 of FIG. 8A for example), such as the type commonly found in an operating rooms, may be used to generate the required suction force (partial vacuum). In some cases, the suction only embodiment of the surgical air shield may create a cooling effect as a result of the evaporation of fluids in the vicinity of surgical site. In general, the suction only embodiment of the surgical air shield will be used with narrower incision sites, as the dual function embodiment of the surgical air shield can generally accommodate wider incision sites relative to the suction only version.

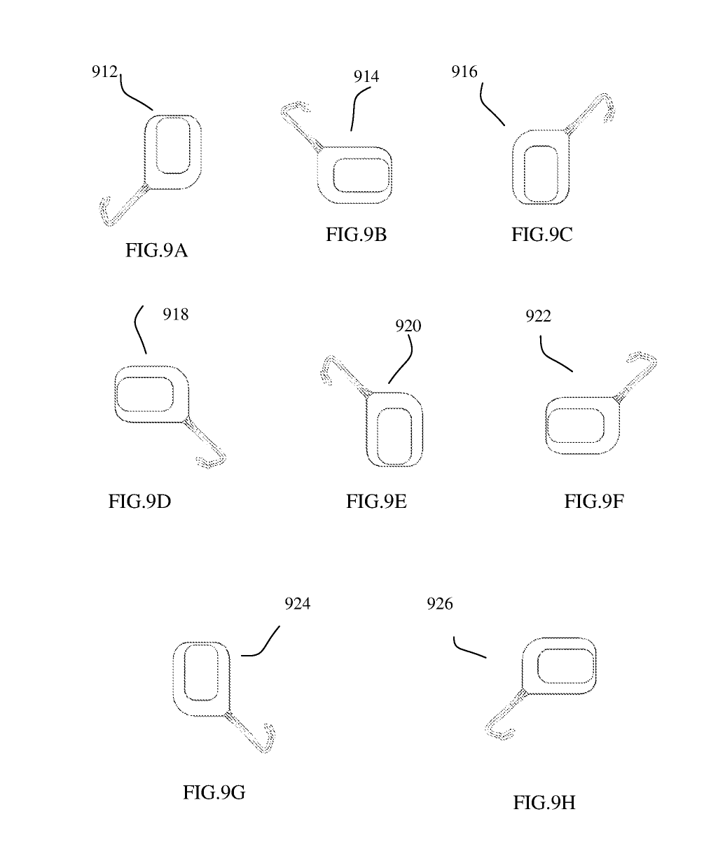

According to an embodiment of the present invention, the surgical air shield is adaptable to be placed in a variety of positions at a surgery site. In a preferred embodiment, placement of the gas shield module is flexible as the module may be designed to have a middle plane of symmetry. As shown in FIGS. 9A through 9H, the gas shield module can be rotated to any desired position/orientation 912, 914, 916, 918, 920, 922, 924 and 926 so that the tubing will extend from any suitable location that will cause minimal interference with the procedure being performed. While in some embodiments the gas shield module may be secured directly to the patient's body, in others the surgical air shield will be secured on the draping system used for the surgical procedure. One of ordinary skill in the art would appreciate that the surgical air shield could be usefully attached in a variety of locations, and embodiments of the present invention are contemplated to be used in any such location. In some embodiments, the surgical gas shield may be disposable, while in other embodiments, the surgical air shield may be reusable.

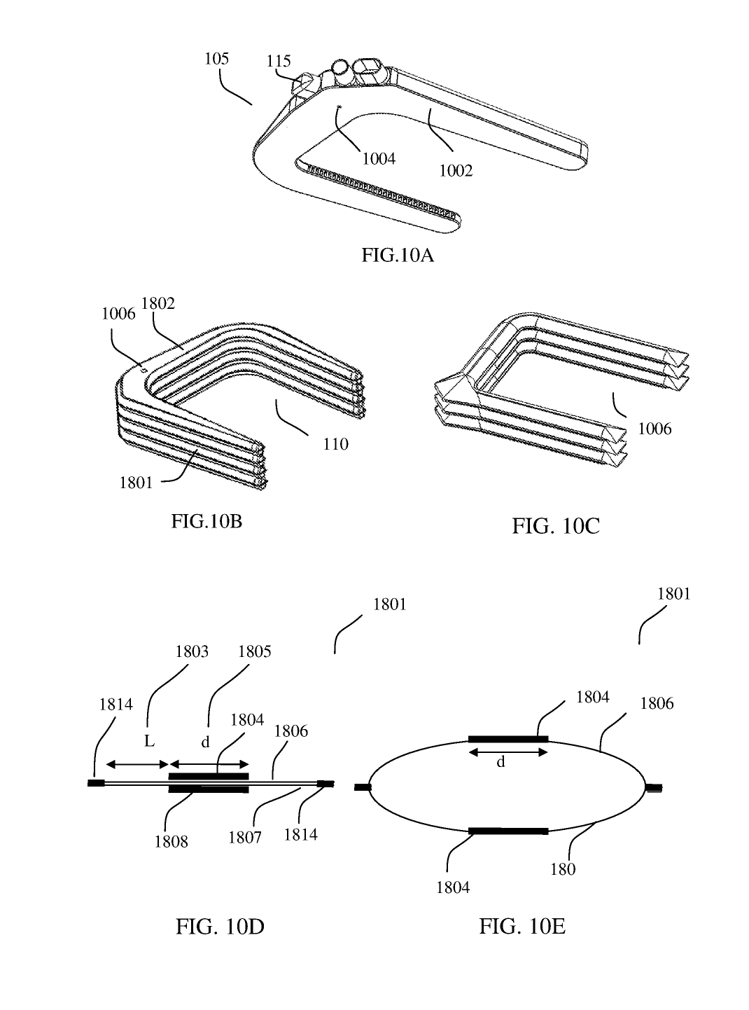

FIG. 10A illustrates a bottom perspective view of a tuning fork type gas shield module 105 which can be mounted on an inflatable type elevation system 110. The gas shield module 105 is provided with an aperture 1004 on the bottom wall 1002. The inflatable elevation system 110 comprises one or more gas cushion elements 1801 stacked one over another. Each of the gas cushion elements 1801 comprises two thin and flexible sheets of any suitable material that is flexible and gas impermeable, including, but not limited to polyethylene, PVC, polypropylene or any other suitable plastic material, rubber, paper or fabric. In the preferred embodiment, the selected material is chosen to withstand the maximum pressure that would be required to raise and withstand the weight of the gas shield module into an elevated position. Reference to FIG. 10D, according to an embodiment of the present invention, two sheets 1806 and 1807 of a gas cushion element 1801 are joined together (exemplary joint 1814 is shown in FIG. 10D) using any suitable bonding means, including, but not limited to, thermal welding, ultrasonic welding, adhesive sheets, or a glue or similar adhesive. In a preferred embodiment, on the top surface 1802 (FIG. 10B) of the uppermost gas cushion element one or more vents/apertures 1006 may be provided. The one or more vents 1006 connect, in fluid communication, the uppermost gas cushion element with a gas shield module mounted on the elevation system. The apertures 1004 and 1006 are provided in such a way that, when the gas shield module 105 is mounted on the elevation system 110, the apertures 1004 and 1006 align with each other. Also, the one or more gas cushion elements of the inflatable elevation system remain in fluid communication with each other among themselves. Thus, as per requirement fluid can transmit in and out of the inflatable elevation system from and to the gas shield module mounted on the inflatable elevation system.

According to a preferred embodiment of the present invention, the gas cushion can be made using two identical paper, rubber, plastic or fabric sheets. In an alternate preferred embodiment, the gas cushion can be made from a single sheet folded in a particular manner. Furthermore, the gas cushion can be made using commercially available bellows made in a variety of different materials such as plastic, rubber or fabric.

FIG. 10C illustrates a top perspective view of a stacking arrangement of another embodiment of the inflatable elevation system of the surgical gas shield device. The dimension and design of the inflatable elevation system depend on many of factors. FIG. 10D illustrates a cross-section of a cushion element 1801 of an elevation system 110 or 1006 (FIGS. 10B and 10C) in deflated condition. The gas cushion element 1801 is in contact with two other gas cushion elements in the stack above and below it and the width “d” of the contact surfaces 1804 with the cushion elements above and below the cushion element 1801 is marked as 1805 in FIG. 10D. The two walls (1806 and 1807) of the cushion element 1801 are welded or joined together by any suitable means at the ends. The width “L” between the contact surfaces 1804 and joints 1802 is shown as 1803. When gas cushion elements of inflatable elevation system are fully pressurized, the shape of the elements is dictated by cushion geometry and wall stresses (as the elements try to assume the shape of a full circle subject to flat contact surface constraints). The maximum height of elevation achieved by the elements will be primarily determined by the distance “L” between the welds and the contact interface.

However, as illustrated in FIG. 10E, when the gas cushion element 1806 is partially pressurized, the elevation and shape of the gas cushion element get adjusted to achieve static equilibrium. This leads to the result that, during the process of inflation, at a particular short lengthwise element section, since the pressure remains same throughout the gas cushion element, the contact width “d” indicated by 1805 should be proportional to the weight of the gas shield module for that length of the gas shield module in that particular gas cushion element. Consequently, parameter “L”, the width between the contact area and the welds, determines the height the gas cushion element attains under a given pressure. Height of the elevation system increases if “L” is increased and vice-versa.

According to an embodiment of the present invention, the shape of the gas cushion elements and the distance between the joints 1814 can have a variety of values. In a preferred embodiment, it is possible to make the width variable with the aim of having a gas cushion that would result in elevation to a plane that is inclined at an angle to the original plane of the platform when the gas cushion is deflated. As shown in FIG. 10B and FIG. 10C, the dimensions of the gas cushion elements of the elevation systems 110 or 1006 can be shaped to suit a particular application so that the final desired elevation is achieved. One of ordinary skill in the art would appreciate that there are many ways to stack and arrange the gas cushion elements for proper height adjustment for a particular application, and embodiments of the present invention are contemplated for use with any such arrangement of gas cushion elements.

Similarly, for a constant rate of elevation of gas cushion elements with pressure (in other words, on a plane that is parallel to the original plane of the elevation system when the gas cushion elements are deflated), the difference in width between the contact area between consecutive gas cushion elements and the welds should be constant. In general, the gas shield module 105 of FIG. 10A has variable weight distribution along its body. As illustrated in FIG. 10B the shape of the gas cushion elements of the elevation system 110 is accordingly designed to support the variable weight distribution of the gas shield module 105. The profile of the adhesive sheet or joined surfaces of the gas cushion elements match the profile of the gas shield module 105 while keeping the width “L” constant throughout the area of contact in a preferred embodiment.

Therefore, when designing the gas cushion elements and the shape of the adhesive/contact area between them, consideration will have to be made for the weight distribution of the gas shield module which the cushion supports. Knowing the weight distribution of the gas shield module would therefore enable the shape of the gas cushion elements or, to that effect, dimension of the elevation system, to be determined for a desired inclination of elevation with pressure.

In some embodiments, in an inflatable type of elevation system, a single valve or actuator can be used for pressurizing or depressurizing the inflatable elevation system of the present invention. In such embodiments, only one aperture is needed in the gas shield module as well as in the gas cushion element for transmission of fluid between the gas shield module and the gas cushion elements.

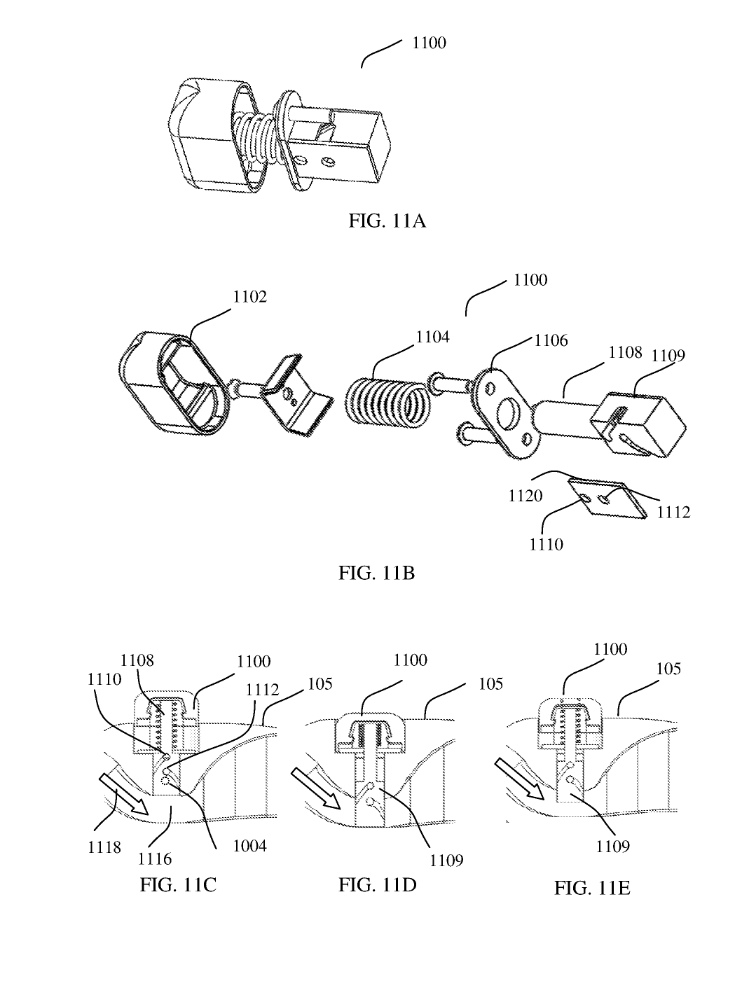

FIG. 11A illustrates a push type valve (the terms “valve”, “regulator” and “actuator” are used alternatively and interchangeably) 1100. As shown in FIG. 11B, the push type valve 1100 comprises a knob 1102, a spring 1104, a mounting bracket 1106, a valve body 1108, a valve head 1109 and a seal 1120. The mounting bracket 1106 allows to secure the valve 1100 to the positive pressure flow channel 1116 in the body of the gas shield module 105 as shown in FIG. 10A. In idle condition, the spring 1104 forces the valve body 1108 to stay away from the flow channel 1116 without obstructing the flow of gas in the channel 1116 as shown in FIG. 11C. Arrow 1118 shows the direction of flow of gas to the gas shield module 105 through the channel 1116. A user can manually press the knob 1102 to push the valve body 1108 down in to the flow channel 1116 as shown in FIG. 11D which obstructs the flow of gas in the channel 1116. The seal 1112 is a simple pad that comprises of two apertures 1110 and 1112 which correspond to the apertures provided on the valve head 1109. At a completely pushed down condition, as in FIG. 11D, the upper aperture 1110 aligns with the aperture 1004 (aperture 1004 is also shown in FIG. 10A) provided on the body of the gas shield module 105. In this state, the pressurized gas flowing in the channel 1116 finds way to the inflatable elevation system attached underneath the gas shield module 105 through the aperture 1110 of the valve body 1109 and aperture 1004 of the gas shield module 105 to inflate the gas cushion of the elevation system 110 (shown in FIG. 10B). If the push valve 1100 is pressed half-way down, the valve head 1109 slides across the flow channel 1116 only partially and the aperture 1112 aligns with the aperture 1004 of the gas shield module 105 as shown in FIG. 11E. In this state, the flow path between the elevation system and the flow channel 1116 gets connected to the downstream side of the flow which gets throttled by the partially obstructing valve head 1109. The throttling effect makes a pressure difference between the inflated gas cushion side and the downstream of flow channel 1116 side with the pressure in the flow channel 1116 being lesser than the elevation system 110. This makes the gas from the inflated elevation system flow back into the flow channel 1116 deflating the gas cushion elements of the elevation system 110 and therefore lowering the gas shield module.

In the present embodiment, the valve components are made of suitable plastic materials but can obviously be made of any suitable materials that meet cost and production requirements. The sealing pad 1120 should preferably be made of a soft material that can perform the sealing action and has a low friction coefficient. This would reduce the force needed to actuate the valve and the stiffness of the corresponding spring 1104.

The valve 1100 can be sized to suit the size of the shield module in which it is used. The pressure transmission hole can be made of such a size to reach a compromise between the speed of actuation and sealing requirements. In alternative embodiments, the valve 1100 may be actuated electrically, remotely or by any other suitable means.

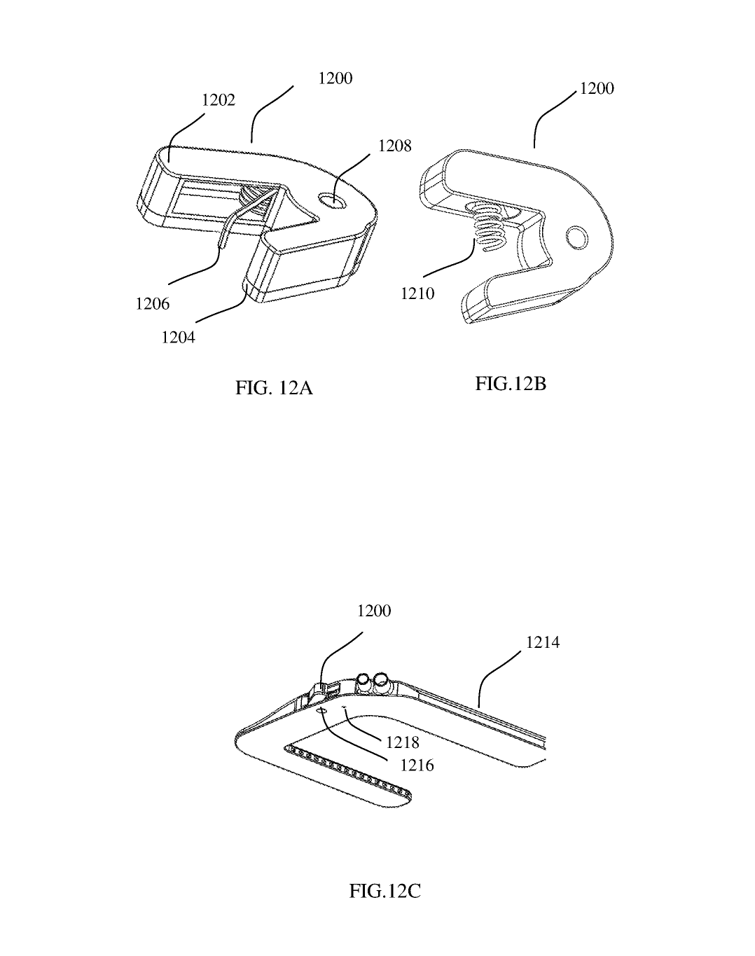

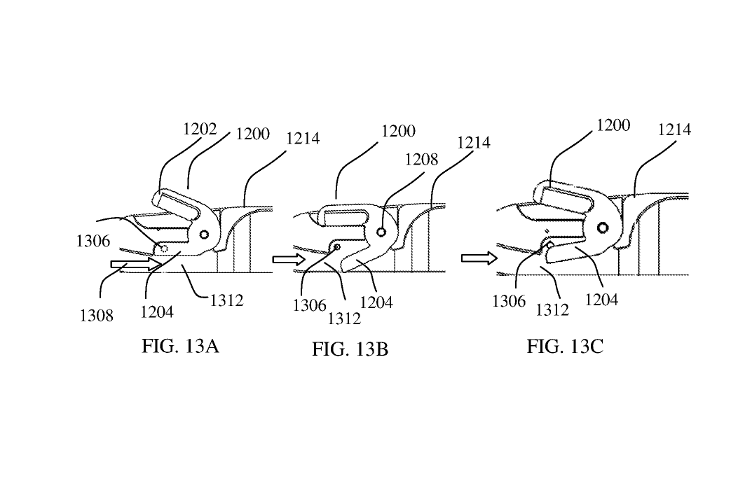

In another embodiment, a single rocker type valve 1200 is provided in the gas shield module of first embodiment surgical gas shield device to control gas flow between the gas shield module and the gas cushion elements of inflatable elevation system similar to the way push type valve 1100 does. The rocker type valve 1200 comprises an actuation arm 1202, a sealing arm 1204, and as shown in FIGS. 12A and 12B, a torsion spring 1206 or a compression spring 1210 used to make the rocker type valve 1200 return to the original idle position as shown in FIG. 13A. The actuation arm 1202 and the sealing arm 1204 together make a substantially V-shaped or U-shaped body with an aperture 1208 being provided in the body where the actuation arm 1202 and the sealing arm 1204 meet each other. The rocker type valve 1200 is pivotally attached to the gas shield module 1214 with the help of a fastener passing through the aperture 1208 of the rocker type valve and aperture 1216 of the gas shield module 1214. At idle position, as shown in FIG. 13A, the actuation arm 1202 juts out of the gas shield module 1214 and the sealing arm 1204 remains clear of the gas flow channel 1312 through which pressurized gas flows in the direction 1308. At this position of the rocker type valve 1200, the sealing arm 1204 completely covers and seals the aperture 1306 provided on the body of the gas shield module 1214. The aperture 1218 is provided, as shown in FIG. 12C, on the side of the gas shield module 1214 to which the gas cushion elements of the elevation system are attached. Thus, when the actuation arm 1202 is pressed down, as shown in FIG. 13B, the sealing arm 1204 pivots around 1208 and obstructs the flow channel 1312 while exposing the aperture 1306 to the upstream gas flow in the flow channel 1312. The pressurized gas flows to the gas cushion element attached to the gas shield module 1214 through the aperture 1306 and inflates the gas cushion of the inflatable elevation system 110 (inflatable elevation system 110 shown in FIG. 10B. When the rocker type valve 1200 is pressed partially, as shown in FIG. 13C, the sealing arm 1204 throttles the gas flow in the flow channel 1312 and the reduced flow pressure in the downstream makes the gas flow back from the inflated gas cushion elements to the flow channel 1312 through the aperture 1306 deflating the gas cushion and lowering the gas shield module.

The valve 1200 can be sized to suit the size of the gas shield module in which it is used. The pressure transmission hole/aperture 1306 can be made of such a size to reach a compromise between the speed of actuation and sealing requirements. In alternative embodiments, the valve 1200 may be actuated electrically, remotely or by any other suitable means.

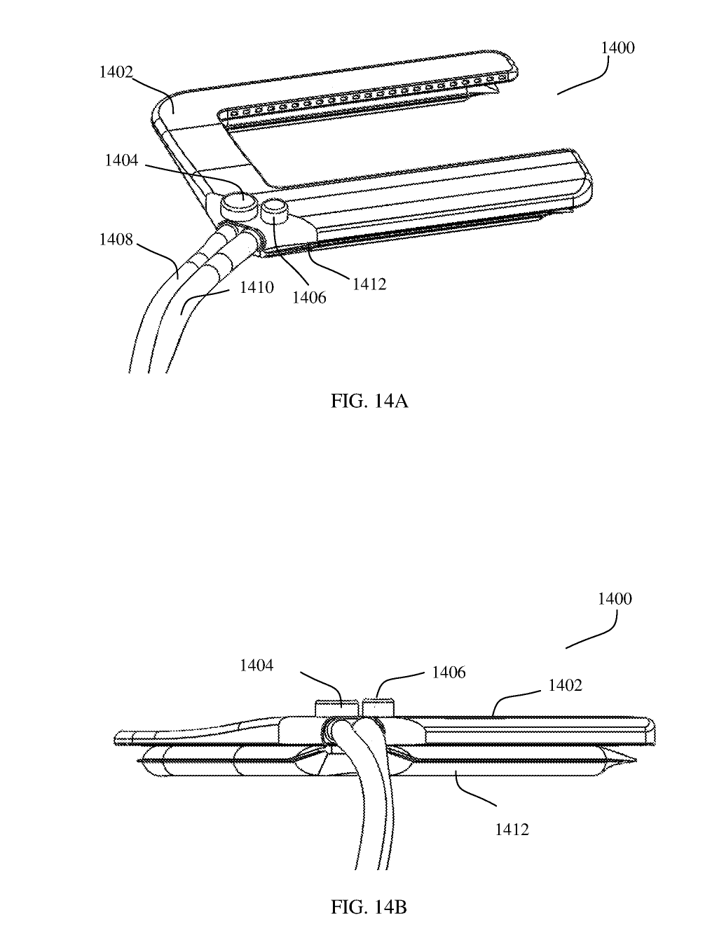

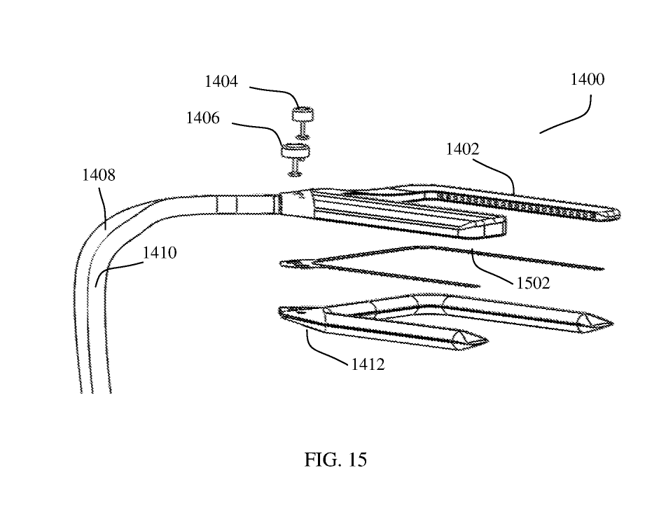

Reference to FIG. 14A and FIG. 14B, in one embodiment 1400 of the surgical gas shield device, two separate regulating valves 1404 and 1406 can be provided in the discharge chamber (connected to the positive pressure line 1408) and suction chamber (connected to negative pressure line 1410) of a gas shield module 1402 respectively. Inflation or deflation of the elevation system 1006 can be controlled by the valves 1404 and 1406 respectively. Exploded view of the surgical gas shield device 1400 illustrated in FIG. 15 shows an exemplary adhesive layer 1502 which can be used for joining the gas shield module 1402 with the inflatable elevation system 1412.

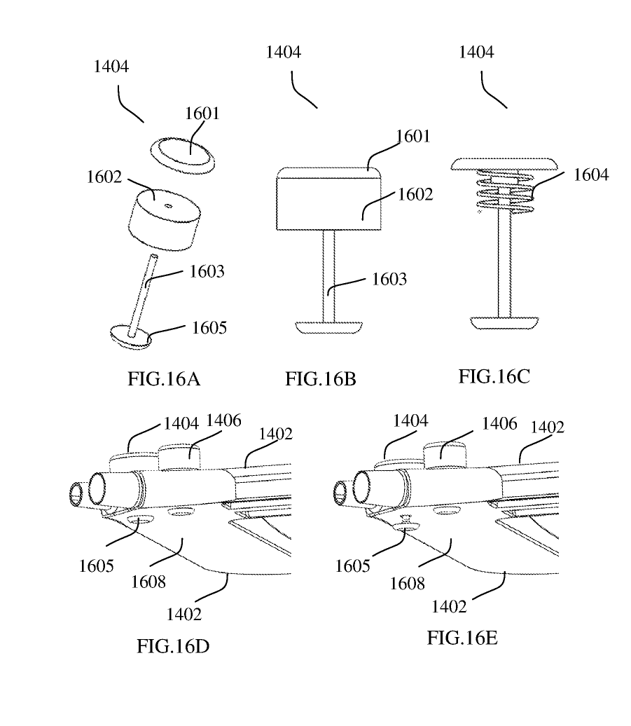

FIG. 16A illustrates exploded view of the valve 1404 used with the gas shield module 1402 of FIG. 15 to actuate inflation and deflation of the gas cushion, in accordance with an embodiment of the present invention. Although, only one valve 1404 is shown in FIGS. 16A, 16B and 16C, it is to be understood that, structurally and functionally, valve 1406 is similar to valve 1404 with probable difference in dimensions only. In one embodiment, the valve 1404 may comprise a button 1601, a spring material 1602, a spindle 1603 and a valve seat 1605, as shown in FIG. 16A. These components can be made of any suitable material, including, but not limited to, metal or plastic. As shown in FIG. 16D, in a preferred embodiment, the spindle 1603 passes through the positive or negative pressure regions of the gas shield module 1402 and the seat 1605 seals an outlet/aperture/vent provided on the lower wall 1608 of the gas shield module 1402 from outside. When the valve button 1601 is pressed, as shown in FIG. 16E, the spindle 1603 is lowered and the valve seat 1605 moves down to expose the vent provided on the gas shield module 1402 to the adjoining gas cushion element of the elevation system. This allows transmission of fluid between the gas cushion elements of the elevation system and the gas shield module. In a preferred embodiment, the valve button 1601 is manually pressed for activation. In alternate embodiments, the valve button may be actuated electrically, remotely or by any other suitable means.

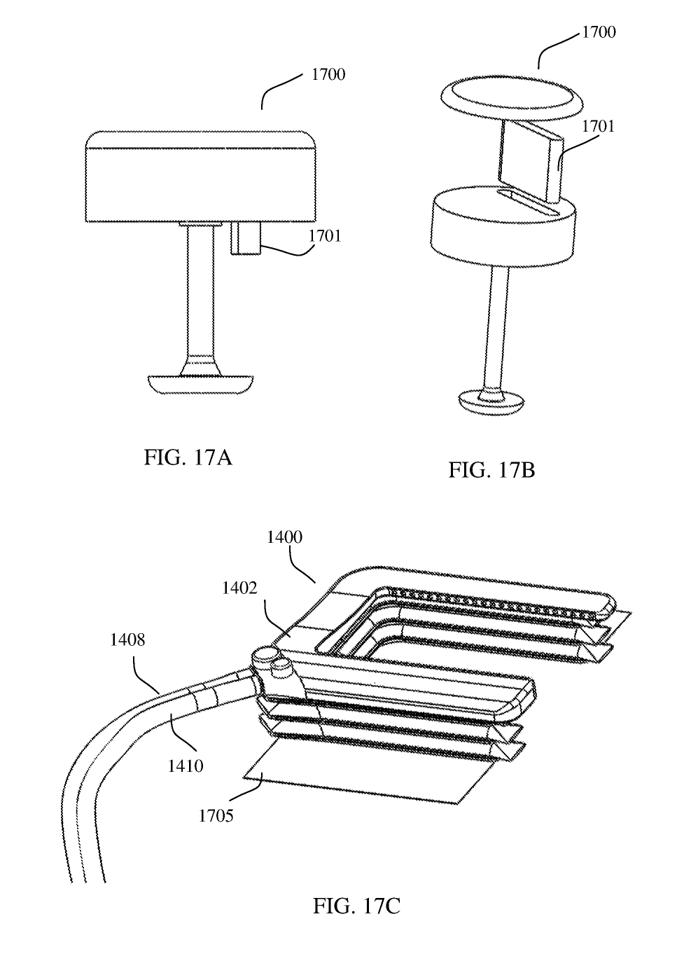

In situations where the pressure resistance in the gas shield module is low and the pressure generated inside the elevation system to inflate the gas cushion elements is not sufficient, a number of solutions can be used. An illustrative example of one such solution is shown in FIG. 17A, which is designed to increase the pressure available to inflate the gas cushion elements by blocking the flow of gas to the surgical gas shield. As shown in FIG. 17A, the valve 1700 is provided with a blocking component 1701. In a preferred embodiment, the blocking component 1701 is arranged to move up and down to block the flow of gas feeding the gas shield module in the internal flow channels. In an alternate embodiment, the main pressurizing region blocking component 1701 can be arranged to rotate and block the flow of gas feeding the gas shield module. In a preferred embodiment, to reduce the possibility of gas leaks from the gas cushion element, when pressurized, flexible washers can be added at selected, critical locations.

According to an embodiment of the present invention, attachment of the surgical gas shield device to the body of a patient can be made more secure by using an extended area of adhesive tape of sheet. This is illustrated in FIG. 17C wherein an extended adhesive sheet 1705 is provided with the surgical gas shield device 1400 which can be used to detachably attach the device at a desired place.

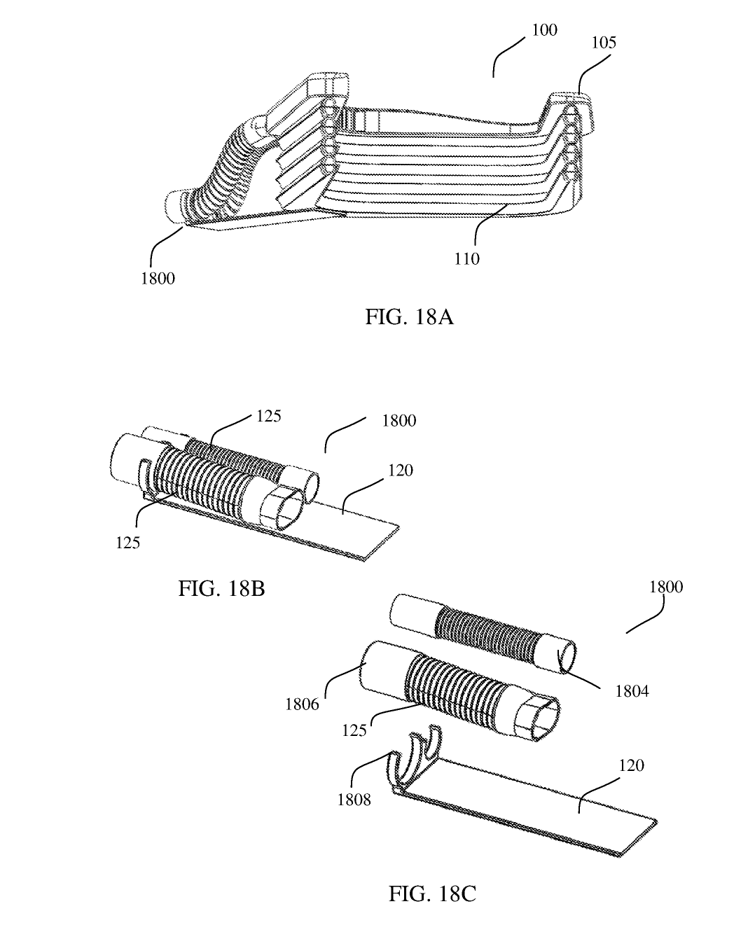

One problem associated with the use of the surgical gas shield device is the pull (or push in some cases) exerted by the inlet outlet tubes on the gas shield module and elevation system assembly while in operation. For example, while inflating or deflating the elevation system to raise or lower the gas shield module 1402 of the surgical gas shield device 1400, as shown in FIG. 17C, the gas shield module 1402 may get pulled by the weight of the tubes or by the jerks exerted due to change in pressure in the tubes. As a solution, the present invention provides an anchorage assembly 1800 as shown in FIG. 18A. The anchorage system 1800 is designed to mechanically isolate the tubing from the gas shield module 105 and the elevation system.

As shown in FIG. 18B and in the exploded view of the anchorage system 1800 in FIG. 18C, the anchorage system 1800 comprises an anchorage block 120 which can be made of plastic or any suitable material and two flexible tubes 125 of required dimension provided with proper end fittings or adaptors (e.g. 1804 and 1806). During assembly of the system, the plain, flat end of the anchorage block 120 is attached to the lower end of the elevation system, as shown in FIG. 18A, which is to rest on the patient's body. For use, first ends of the adaptors are fitted to the corresponding inlet/outlet ports of the gas shield module 105 as shown in FIG. 18A and the other ends of the adaptors are fitted to the pressure and suction lines (not shown in the FIG.) while secured to the anchorage block 120. There can be many ways available for securing the adaptors to the anchorage block 120. In one preferred embodiment, the attachment is in the form of a snap fit semi-circular collar 1808. Alternatively, more secure attachment can be achieved by snap fitted collars or by collars secured using gluing, adhesives or a variety of mechanical fasteners.

As the elevation of the gas shield module changes, the flexible tubes allow the gas shield module 105 to rise and fall while absorbing the lengthwise dimensional changes. The anchorage mechanism 1800 makes sure that the weight or pull/push exerted by the pressure and suction lines/tubes do not affect the performance of the gas shield module/gas cushion/flexible folds as the anchorage system does not allow the pull/push to get transmitted to the surgical gas shield device.

In designing the inflatable elevation system, it is important to take into account the Pressure-Flow (or P-Q) characteristics of the pressurizing system. Axial fans, centrifugal fans and positive displacement pumps have widely differing characteristics. For example, as the one or more gas cushion elements of the elevation system begin to fill with gas near the position of maximum elevation, the flow rate decreases (and ultimately reaches a value of zero) and the supplied pressure increases. Additionally, when using positive displacement pumps, the maximum pressure may be significantly higher than the maximum pressure supplied by axial fans. This pressure may be so high that it may rupture the gas cushion element material. In a preferred embodiment, either a relief valve can be used or the nature and thickness of the material of the gas cushion can be appropriately selected to accommodate that increased pressure.

According to a preferred embodiment of the present invention, a compression device (positive pressure source), such as a pump or air compressor, can be used to generate the pressure to inflate the gas cushion elements and elevate the gas shield module. In some embodiments, compressed gas canisters or cylinders can be used to inflate the gas cushion elements. In some embodiments, hand pumps may be used for inflation of the gas cushion elements. In some embodiments, multiple types of compression devices, gas canisters, and hand pumps may be used for system redundancy. One of ordinary skill in the art would appreciate that there can be available many suitable devices for inflating the gas cushion elements and embodiments of the present invention are contemplated for use with any such inflation tools.

According to a preferred embodiment of the present invention, the gas used to inflate the gas cushion is compressed air. One of ordinary skill in the art would appreciate that there are many suitable gasses that could be used to inflate the gas cushion and embodiments of the present invention are contemplated for use with any such gas.

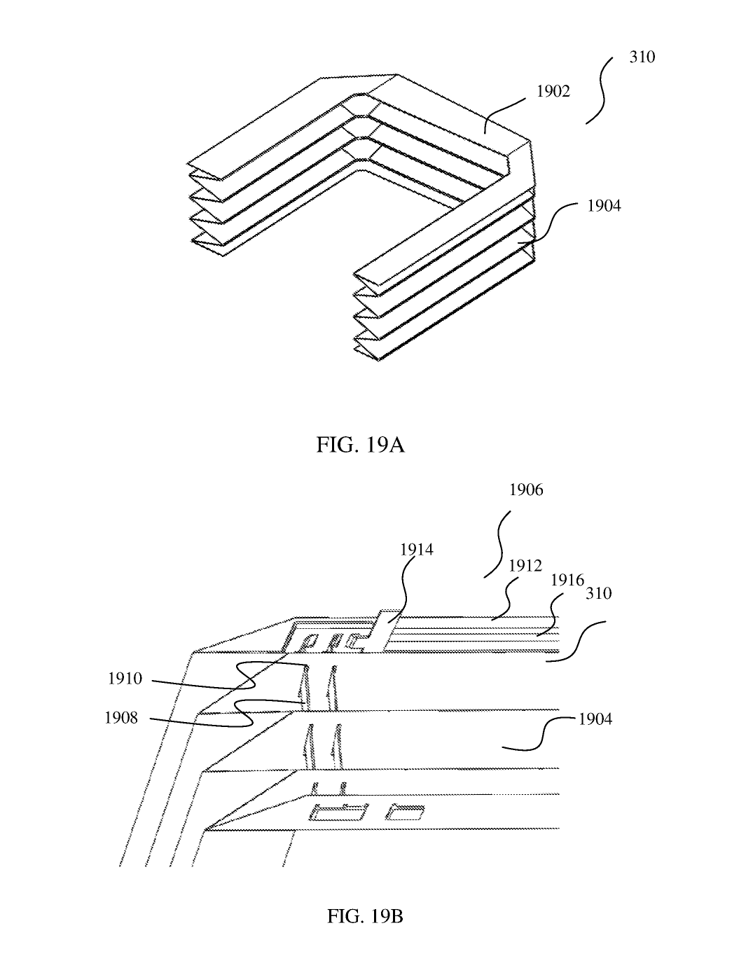

In some preferred embodiments of the present invention, the elevation system enables elevation of the gas shield module to a certain height and/or at an angle of inclination, if required, without using inflatable elevation system. This allows the same surgical gas shield device design to be used in a wider range of surgical procedures such as cavity, skin and breast surgeries. Reference to FIG. 19A, in these embodiments, the elevation system 310 (also referred to as flexible fold elevation system) comprises one or more zigzag-shaped or z-shaped paddings or flexible folds or bellows 1904 (hereinafter referred to as flexible fold 1904) and, optionally, one or more positioner mechanisms (positioner mechanisms are depicted in FIGS. 19B, 20A through 20D and 21A through 21E). The positioner mechanisms enable lockable and releasable adjustment of height and inclination of the elevation systems. The elevation system can be locked at a certain desired height and, then, be released, optionally, from that height and inclination as per requirement with the help of the positioner mechanisms. The material used for the flexible fold 1904 should have dimensional stability and should be flexible, yet sufficiently stiff or rigid to maintain an expanded shape even under the load of a gas shield module mounted on it and keep the flexible fold 1904 from sagging or collapsing after the folds are expanded. Alternatively, other elements (such as some of the positioning elements described below) can support the expected loads in which case, the folded material can be selected to be thinner and more flexible. The flexible fold 1904 can, for example, be made using any method of folding or forming such as vacuum forming, casting or joining of plastic sheets by application of direct heat, ultrasound or by any other suitable means. They can be made from a number of sheets joined together or from a single sheet folded in “accordion fashion”. The width of the folds and the number of folds can evidently be selected to suit the initial and final desired height of the elevation system.

The one or more positioner mechanisms can be provided at specific locations around the periphery of the flexible fold 1904 of the elevation system 310. FIG. 19B shows a first embodiment of the flexible fold elevation system. The individual folds can be provided with suitably sized slots 1910 aligned along a vertical axis through which a strip 1908, hereinafter also referred to as positioner strip 1908, can be inserted from the top and loop around the lowermost fold and then returned back to the top fold 1912 through the slots with the loose ends of the strip remaining attached to a pull tag 1914 or, conversely, using a single, folded strip with the loose ends joined together at the lowermost fold. The positioner strip 1908 can be made of any suitable material such as Polyethylene, Polypropylene, PTFE, PVC, etc. and are able to provide the desired stiffness and flexibility. In addition to the material selected, several additional factors assist in providing the desired combination of stiffness and flexibility. These include the strip width and thickness. For additional vertical stiffness, two or more such strips can be used at the same general location, effectively providing correspondingly multiple pillars to support vertical forces. The lower end of the single or multiple strips can be attached to the lowermost fold of the elevation system using mechanical fasteners, adhesives or thermal welding.

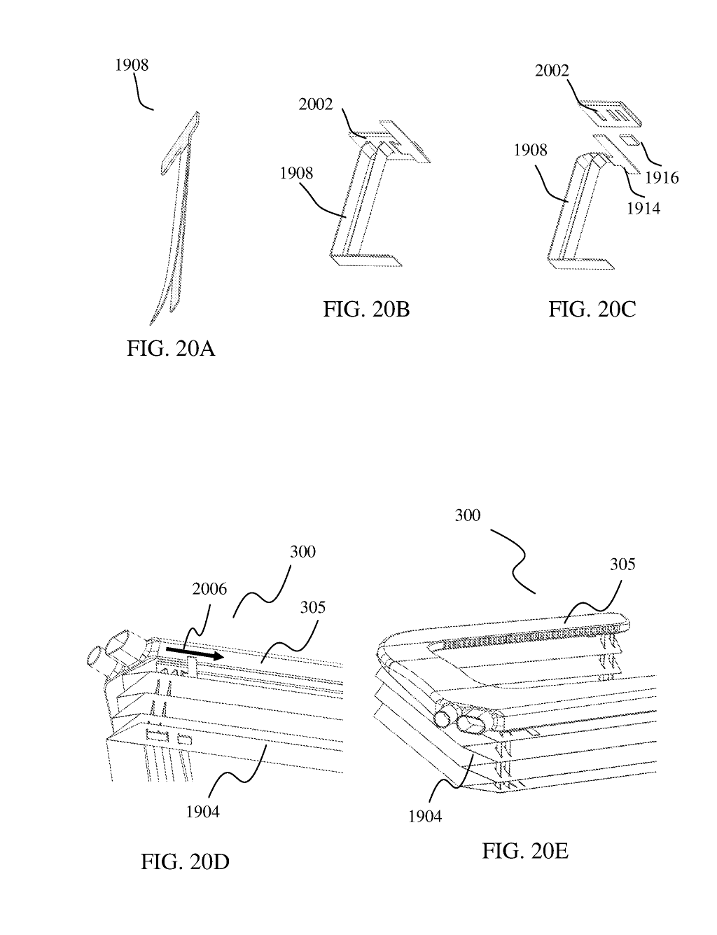

FIG. 20A shows an exemplary embodiment of the positioner strip 1908 which can be a folded form of an otherwise straight piece of strip. FIGS. 19B, 20B and 20C show as to how the pull tag 1914 of the positioner strip 1908 can be detachably attached beneath the uppermost fold 1912 with the help of a sleeve 2002 after looping around the flexible folds.

When the positioner strip 1908 is pulled with the help of pull tag 1914, preferably along the length of the fold in the direction 2006, the flexible fold 1904 is forced to collapse and the height of the elevation system is decreased lowering the gas shield module mounted on it. Pulling the tag 1914 in the opposite direction raises the elevation system. With several such positioner mechanisms located at strategic positions in the flexible folds 1904 of the elevation system, any desired height and inclination can be achieved.

The strip end or pull tag 1914 can be secured to a desired position using any possible releasable attachments. Examples of releasable attachments include hook and loop fasteners, a small magnet and a magnetic strip combination, rubber or other flexible plastic piece engaging a suitably sized longitudinal or lateral recesses or grooves, spring-loaded clips, snap-on fasteners, hook-and-loop fasteners or re-closable fasteners or tapes, etc. Reference to FIG. 19B and FIG. 20C, re-sealable tape 1916 is one such releasable attachment used for detachably securing the tag 1914 to the flexible folds.

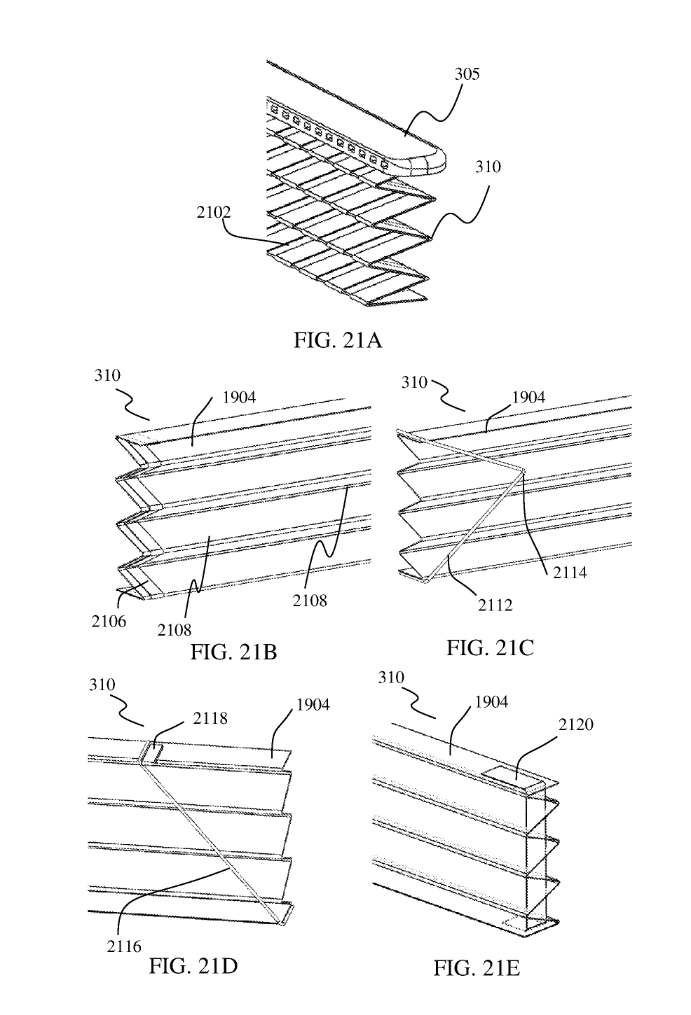

Various other embodiments of the positioner mechanism for controlling the elevation and inclination of the flexible fold type elevation system of the present invention are illustrated through FIGS. 21A to 21E. It is evident that some of these suggested mechanisms can be applied to both the flexible fold type and inflatable (gas cushion) type elevation systems.

Desired level of rigidness of the flexible fold elevation system can be achieved through use of ribbed material 2102 in the folds as shown in FIG. 21A. The laterally flexible material 2102 in FIG. 21A can be constructed using commercially available suitably ribbed plastic materials with alternating thin and thicker sections to provide the rigidity needed for vertical flexibility and the lateral flexibility needed to accommodate the lateral variations in the body contours. Alternatively, the material 2102 can be constructed by sandwiching or laminating a material with good springiness, such as cellulose acetate for example, between two thin layers of a very flexible material such as polyester, polyethylene or plasticized PVC sheets and the whole assembly can be welded thermally or ultrasonically or using any other suitable means. Other suitable materials include, but are not limited to, Polyethylene, Polypropylene, Acetal copolymer, Linear Polyphenylene sulfide, Nylon and ABS. In an alternative embodiment, the structural rigidity of the system can be provided by the positioning strips only, in which case, a thinner and more flexible material can be used for the folding system to provide only a barrier to the surgical site.

In the embodiment shown in FIG. 21B, one or more deformable thin strips 2106 are attached to the folds 1904 orthogonally to the fold lines 2108 at the edges or at places evenly or unevenly spaced apart. When pressed manually by a user, the one or more strips 2106 deform at the fold lines 2108 and hold the flexible folds at the desired position. The gas shield module can be positioned at a plane inclined at any desired angle to the plane of the surgical site when one or more deformable strips 2106 are pressed down by different distances.

FIG. 21C illustrates another embodiment of the flexible fold elevation system in which a deformable element 2112 is attached to the uppermost and lowermost strips of the flexible fold 310. The material itself can be deformed or a joint 2114 can be a made as a bent part of the element 2112 or it can be made in the form of a friction hinge with or without position indexing. When the flexible fold 1904 is pushed downward, the deformable element 2112 bends around the joint/hinge 2114 and stays at that position forcing the flexible fold 1904 to take the desired position at that location. A number of such elements 2112 strategically located around the perimeter of the flexible fold 1904 yields the desired control over elevation and inclination of the elevation system 310.

In another embodiment of the flexible fold type elevation system, as shown in FIG. 21D, a slender element 2116 is attached to the flexible fold 1904. The lower limb of the element 2116 is rotatably secured to the lowermost fin/strip of the folds. The upper limb of the element 2116 is rotatably and slidingly secured to the uppermost fold with the help of a lockable mechanical slide 2118. When the flexible fold is pushed down, the element 2116 tilts. The tilting of the element 2116 is facilitated by the rotation of the lower limb of the element 2116 and sliding of the upper limb of the element. After reaching a desired location, the mechanical slide 2118 can be prevented from sliding backward so that the flexible fold remains at that elevation. However, the mechanical slide 2118 can be allowed to slide further forward in case the flexible fold 1904 gets unintentionally pressed down again. In such cases the slide 2118 will come back to its desired position once the pressure is withdrawn. A number of such elements can be provided to the elevation system to give the desired control of elevation and inclination of the elevation system 310.

In yet another embodiment of the flexible fold elevation system, one or more pieces of strings or flexible strips 2120 are disposed with at the ends of the flexible fold 1904 as shown in FIG. 21E. Each of these strips 2120 can be pulled along the perimeter of the flexible fold 1904 or sidewise from above or below the folds to raise or lower the gas shield module at that location. The string or strip 2120 can be secured at position using mating hook and loop (e.g. Velcro) strips or a spring loaded clipper or a variety of lockable releasable mechanisms. With a minimum of four such elements or more, the assembly's elevation and inclination can be adjusted. This arrangement also allows the flexible folds to compress further and then return to the desired position if the assembly is unintentionally pressed down.

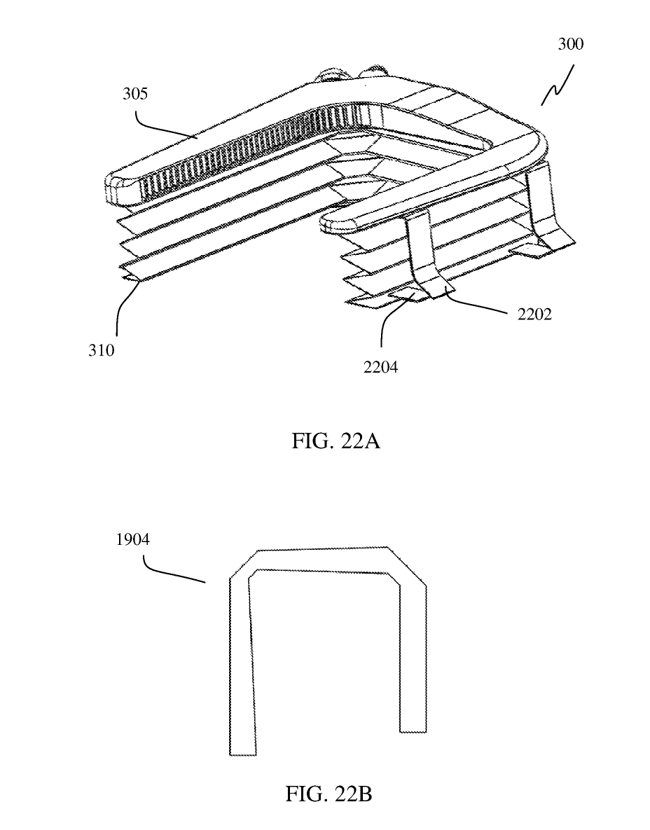

The surgical gas shield device 300 can be secured to a desired position and inclination by the use of adhesive sheets or tape as is normally used in medical procedures or by hook-and-loop elements or by snap-on male and female elements or other means of attachment or re-attachment. This is illustrated in FIG. 22A. To secure the assembly at a position relative to the body of the patient and to maintain the required elevation and inclination, in one possible embodiment one or more strips of adhesive material or hook and loop elements or other means of attachment or re-attachment can be used. In this case, a portion of the adhesive or attachment material 2202 is attached to the top of the gas shield module 305 and the other portion of the adhesive or attachment element 2204 is attached to the body of the patient or to the draping system. In a preferred embodiment, the adhesive or attachment material or element supplied is already attached to the assembly and the user simply pushes the assembly from above and secures the lower part of the adhesive or attachment material or element to the body of the patient or the draping system.

The design of the flexible fold 1904 with the positioning mechanism has to take care of the weight distribution of the gas shield module and the various forces expected during use so that the gas shield module can be positioned at an acceptable or desired initial position. Some of the important design considerations for flexible fold 1904 are, but not limited to, the size and weight of the gas shield module, forces expected to be exerted on the system during use, vertical flexibility of the folds, nature and thickness of material used for the folds, width of the folds (generally, the wider the folds, the greater the flexibility) etc. FIG. 22B illustrates top view of an exemplary profile of a flexible fold 1904 which has been designed by taking the above mentioned design criteria into account for a certain gas shield module.

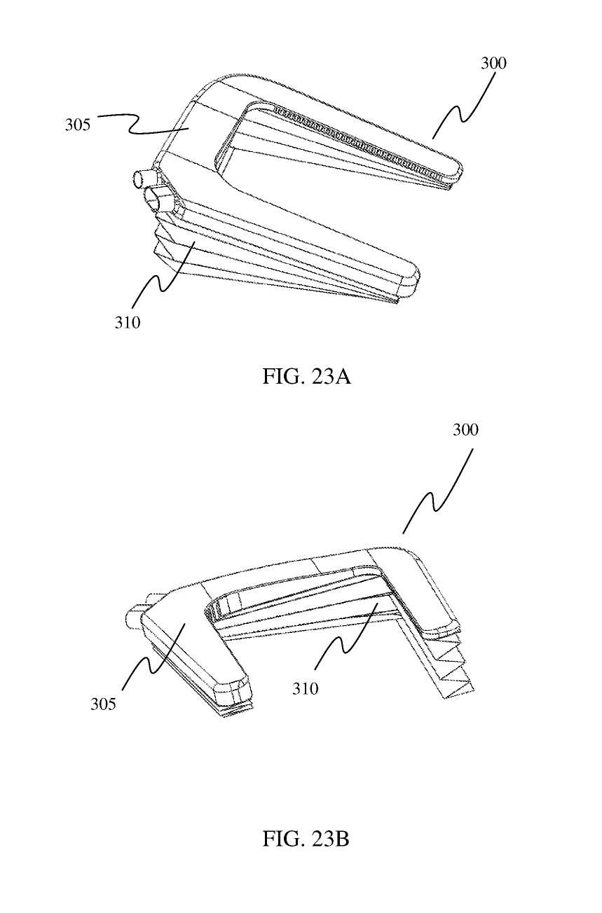

FIG. 23A and FIG. 23B show as to how a flexible fold elevation system 310 disposed with one or more of the above mentioned positioner mechanisms enable positioning of the gas shield device 305 at any desired plane elevated from the base and adjusted parallel or inclined to the base. As illustrated in FIG. 23A and FIG. 23B, the gas shield module 305 can be pushed manually from above to compress the flexible folds of the elevation system 310 to get the required elevation or inclination of the gas shield module 305. In one embodiment, the flexible fold 1904 is resilient and tends to regain its original shape in the vertical direction once the pressure or load over it is removed.

Using corrugated or folded sheets is only one of the methods of constructing the flexible folds. It is also possible to use soft, deformable materials such as polyester padding to achieve the same result. It can even be envisaged to construct such a system using a series of springs in parallel. Other possible methods may use stacks of flexible pieces of tubing to achieve both the vertical and the lateral flexibility mentioned above. The flexible folds can be sized to suit the size of the gas shield module in which it is used.

The flexible fold design of the elevation system influences the design of the gas shield module also as the necessity of having a valve for controlling gas flow, as in the case of gas cushion type of elevation system, is done away with.

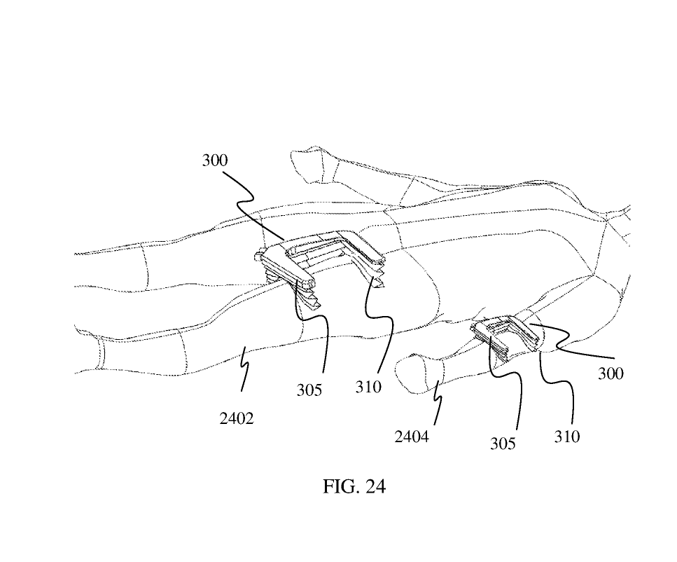

The flexible fold elevation system conforms closely to the natural contours of body parts and, thus, it allows positioning of the surgical gas shield device 300 at any uneven place on the patient's body. FIG. 24 shows two surgical gas shield devices 300 of different dimensions placed at two different places on a patient's body. A bigger surgical gas shield device 300 is shown placed on the leg 2402 of a patient whereas a smaller surgical gas shield device 300 is shown placed on the arm 2404 of the patient.

According to an embodiment of the present invention, the system encompassing the invention can also be used without elevating the gas shield module by partially inflating the gas cushion elements to provide a soft interface between the patient's body and a rigid surface at the top. In the preferred embodiment, the flexible lower part may also be useful in allowing the system to accommodate different contours and curvatures of the patient's body.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The terms “affixed”, “fitted”, “attached”, “tied” are to be construed as partly or wholly contained within, attached to, or joined together, even if there is something intervening. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A surgical gas shield device, said device comprising:
a gas shield module, said gas shield module having a body housing, said body housing divided into a discharge chamber and a suction chamber, said discharge chamber configured to be coupled with a positive pressure source and said suction chamber configured to be coupled with a negative pressure source, a plurality of discharge ports disposed along an inner periphery of said discharge chamber and a plurality of suction ports disposed along an inner periphery of said suction chamber; and
an elevation system attached to said gas shield module, said elevation system configured to hold said gas shield module around a surgical site at an adjustable height and inclination with respect to said surgical site;
wherein discharge of a gas introduced from said positive pressure source through said plurality of discharge ports over said surgical site and suction of said gas and a plurality of air borne byproducts from said surgical site through said plurality of suction ports by application of partial vacuum to said suction chamber by said negative pressure source create a gas shield over said surgical site.

2. The surgical gas shield device as in claim 1, wherein said gas shield module is a fork type gas shield module.

3. The surgical gas shield device as in claim 1, wherein said gas shield module is a closed loop type gas shield module.

4. The surgical gas shield device as in claim 1, wherein said discharge chamber and said suction chamber of said gas shield module are provided with one or more flow channels.

5. The surgical gas shield device as in claim 1, wherein said elevation system is an inflatable type elevation system comprising one or more gas cushion elements configured to selectively be in fluid communication with said gas shield module by use of one or more valves provided in said gas shield module.

6. The surgical gas shield device as in claim 5, wherein said one or more valves provided in said gas shield module is a push type valve which enables control of inflation and deflation of said inflatable type elevation system to attain said adjustable height and inclination.

7. The surgical gas shield device as in claim 5, where said one or more valves provided in said gas shield module is a rocker type valve which enables control of inflation and deflation of said inflatable type elevation system to attain said adjustable height and inclination.

8. The surgical gas shield device as in claim 5, wherein design of said one or more gas cushion elements is made to support a non-uniform weight distribution of said body housing of said gas shield module.

9. The surgical gas shield device as in claim 1, wherein said surgical gas shield device further comprises an anchorage system.

10. The surgical gas shield device as in claim 9, wherein said anchorage system comprises two flexible tubes which connect said positive pressure line and said negative pressure line to said gas shield module and an anchorage block that holds said two flexible tubes in place to isolate said surgical gas shield device from any push or pull exerted by said positive pressure line and said negative pressure line.

11. The surgical gas shield device as in claim 5, wherein said one or more gas cushion elements of said inflatable type elevation system conform to contours of a body part of a patient to hold said gas shield module over said surgical site at said adjustable height and inclination.

12. The surgical gas shield device as in claim 1, wherein said elevation system is a flexible fold type elevation system comprising one or more flexible folds.

13. The surgical gas shield device as in claim 12, wherein said one or more flexible folds substantially conform to contours of a body part of a patient to hold said gas shield module over said surgical site at said adjustable height and inclination.

14. The surgical gas shield device as in claim 12, wherein said flexible fold type elevation system further comprises one or more positioner mechanisms that enable a lockable and releasable adjustment of elevation and inclination of said one or more flexible folds to attain said adjustable height and inclination.

15. The surgical gas shield device as in claim 14, wherein said one or more positioner mechanisms comprise one or more positioner strips disposed at one or more locations in said flexible folds, said one or more positioner strips configured to loop around said flexible folds and to provide one or more pull tags below said gas shield module to enable said adjustment of height and inclination.

16. The surgical gas shield device as in claim 14, wherein said adjustment of height and inclination is attainable through application of a manual force on said gas shield module.

17. A method for providing a gas shield at a surgical site, said method comprising:
providing a surgical gas shield device, said surgical gas shield device comprising an elevation system and a gas shield module mounted on said elevation system, said gas shield module having a body housing, said body housing divided into a discharge chamber and a suction chamber, a plurality of discharge ports disposed along an inner periphery of said discharge chamber and a plurality of suction ports disposed along an inner periphery of said suction chamber;
coupling said discharge chamber with a positive pressure source and coupling said suction chamber with a negative pressure source;
placing said surgical gas shield device around a surgical site;
adjusting and readjusting a height and an inclination of said elevation system to hold said gas shield module over said surgical site at a desired position;
introducing a gas supplied from said positive pressure source through said plurality of discharge ports over said surgical site; and
evacuating said gas and a plurality of air borne byproducts from said surgical site through said plurality of suction ports by application of partial vacuum to said suction chamber by said negative pressure source to create said gas shield over said surgical site.

18. The method as in claim 17, wherein said gas shield module is a fork type gas shield module.

19. The method as in claim 17, wherein said gas shield module is a closed loop type gas shield module.

20. The method as in claim 17, wherein said elevation system is an inflatable type elevation system comprising one or more gas cushion elements configured to selectively be in fluid communication with said gas shield module by use of one or more valves provided in said gas shield module.

21. The method as in claim 17, wherein said elevation system is a flexible fold type elevation system comprising one or more flexible folds.

22. The method as in claim 21, wherein said flexible fold type elevation system further comprises one or more positioner mechanisms that enable a lockable and releasable adjustment of elevation and inclination of said one or more flexible folds to attain said desired position of said gas shield module.

23. The method as in claim 17, wherein said adjustment and readjustment of said height and said inclination are attainable through application of a manual force on said gas shield module.

24. A surgical gas shield device, said device comprising:
a gas shield module having a body housing, said body housing comprising a top wall and a bottom wall, said top wall and said bottom wall configured to define one or more chambers when joined together, a plurality of suction ports disposed along an inner periphery of said one or more chambers; and
an elevation system attached to said gas shield module, said elevation system configured to hold said gas shield module around a surgical site on a patient's body and to enable adjustment and readjustment of height and inclination of said gas shield module with respect to said surgical site;
wherein a plurality of air borne byproducts are evacuated from said surgical site through said plurality of suction ports by application of a partial vacuum to said one or more chambers by a negative pressure source detachably connected to said one or more chambers.

25. The surgical gas shield device as in claim 24, wherein said elevation system is an inflatable type elevation system comprising one or more gas cushion elements configured to selectively be in fluid communication with said gas shield module by use of one or more valves provided in said gas shield module.

26. The surgical gas shield device as in claim 24, wherein said elevation system is a flexible fold type elevation system comprising one or more flexible folds configured to conform to an uneven surface around said surgical site.
